(19) United States
(12) Patent Application Publication
(10) Pub. No.: US 2009/0153021 A1
(43) Pub. Date: Jun. 18, 2009

(54) LARGE-BANDGAP HOST MATERIALS FOR PHOSPHORESCENT EMITTERS

(75) Inventors: Kwan-Yue Jen, Kenmore, WA (US); Shi Michelle Lui, Kenmore, WA (US); Yu-Hua Niu, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: WASHINGTON, UNIVERSITY OF, Seattle, WA (US)

(21) Appl. No.: 12/266,987

(22) Filed: Nov. 7, 2008

Related U.S. Application Data
(63) Continuation of application No. PCT/US2007/011300, filed on May 9, 2007.
(60) Provisional application No. 60/798,883, filed on May 9, 2006.

(51) Int. Cl. H01J 1/62; C08G 73/06; C07D 209/82

(52) U.S. Cl. 313/498; 528/422; 548/446

(57) ABSTRACT

Polymers and compounds having high-triplet-energy; guest-host films comprising the polymers or compounds as hosts and phosphorescent compounds as guests; and electroluminescent devices that include the films.

### CROSS-REFERENCES TO RELATED APPLICATIONS

This application is a continuation of International Application No. PCT/US2007/011300, filed May 9, 2007, which claims the benefit of U.S. Provisional Application No. 60/798,883, filed May 9, 2006. Each application is expressly incorporated herein by reference in its entirety.

### STATEMENT OF GOVERNMENT LICENSE RIGHTS

This invention was made with Government support under Contract No. DMR0103009, awarded by the National Science Foundation. The Government has certain rights in the invention.

### BACKGROUND OF THE INVENTION

The performance of organic light-emitting diodes (OLEDs) has improved dramatically over the past decades. In OLED devices, electrons and holes are injected from the opposite electrodes and recombine to form excitons, either singlet or triplet. Only radiative decay of singlet excitons emit light. Because the probability of singlet exciton formation for the devices based on the fluorescent materials is only 25% (based on simple spin-paring statistics), the highest internal quantum efficiency achievable is limited to 25%. The 25% upper-limit can be overcome by incorporating phosphorescent dopants, such as platium, iridium, and osmium organometallic emitters, to harvest both singlet and triplet excitons. Internal quantum efficiency up to 100% can be realized by using triplet emitters. Green-emitting small-molecule-based OLEDs have been demonstrated with nearly 100% internal quantum efficiencies (ηₑₓₜ=19-20%).

Triplet emitters of heavy-metal complexes are normally dispersed in a host material to reduce the quenching associated with the relatively long excited-state lifetimes of triplet emitters and triplet-triplet annihilation. Effective host materials are of great importance for efficient phosphorescent OLEDs. Recent progress in harvesting both singlet and triplet excitons through incorporation of phosphorescent dopants into the organic light-emitting diodes (OLEDs) has led to a significant increase in device efficiency. Both singlet and triplet excitons formed in a host material can be transferred to a phosphorescent dopant and participate in light emission via Förster and Dexter energy transfer processes, thus allowing for up to 100% internal quantum efficiency.

The efficiencies of conjugated polymer-based phosphorescence devices usually are much lower than those of small-molecule-based devices. This reduced efficiency has been attributed to the long effective-conjugation-length that results in a lower triplet energy state. A conjugation length as short as the fluorene trimer has been shown to have a triplet energy level lower than those of blue- and green-emitting phosphors. As a result of the low triplet energy, exothermic energy transfer between the excited phosphor and the triplet state of the fluorene trimer leads to significant phosphorescence quenching. Although external efficiencies of greater than 10% have been demonstrated by blending conjugated polymers with red phosphors, high efficiency polymer-based OLEDs using green- or blue-emitting phosphors as dopants still have not been realized.

### SUMMARY OF THE INVENTION

In one aspect, the invention provides compounds used as hosts for phosphorescent emitters in electroluminescent devices. In one embodiment, the invention provides a polymer having a ground state to singlet excited state energy gap of from about 3.3 eV to about 3.5 eV and a triplet energy greater than about 2.6 eV.

In another aspect, the invention provides a film that includes a compound of the invention and a phosphorescent emitter.

In another aspect, the invention provides an electroluminescent device, including a first electrode, a second electrode, and a film intermediate the first and second electrodes that includes a compound of the invention and a phosphorescent emitter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates polymers of the invention synthesized from monomers containing a meta-linkage between fluorene and phenylene.

FIG. 2 illustrates polymers of the invention synthesized from monomers containing a meta-linkage between phenylene groups.

FIG. 3 illustrates representative branched macromolecules of the invention.

FIG. 4 graphically illustrates the overlap between the PL spectrum of a representative host of the invention, PF-mCzP-mOXDP, and the UV-Vis absorbance of a typical red-emitter guest material, Os-2.

FIG. 5 graphically illustrates an overlay of PL spectrum (circles) and UV-vis absorbance (squares) of a solid-state film of the guest emitter CHY-2r-ppz(CF₃) (Ir-2R).

FIG. 6 graphically illustrates the PL spectra of solid films of Os-2 and PF-mCzP-mOXDP.

FIG. 7 graphically illustrates EL spectra of OLED devices made with films of Os-2 and PF-mCzP-mOXDP as the emissive layer.

FIG. 8 graphically illustrates electroluminescent spectra from OLED devices with the structure ITO/PS-BTPD-PFCB/Ir-2R (guest)-PF-mCzP (host) film/TPBI/CsF/Al.

FIG. 9 graphically illustrates the EL spectra of an OLED device made using an emissive layer of PP-mCzP-mOXDP and a guest emitter, FIrpic.

FIG. 10 graphically illustrates the EL spectra of OLED devices made using emissive layer films of the guest blue-emitter FIr6 incorporated into host materials of the invention MTP-CBP and MTP-CF3-CBP, as well as polyvinyl carbazole (PVK).

FIG. 11 illustrates a representative electroluminescent device of the invention.

FIG. 12 illustrates a representative electroluminescent device of the invention that incorporates a hole-transport layer and an electron-transport layer.

FIG. 13 graphically illustrates the UV-Vis absorption spectra of films of polymers of the invention.

FIG. 14 graphically illustrates the photoluminescence spectra of polymers of the invention.

FIG. 15 illustrates the synthesis of PF-mCzP, a representative polymer of the invention.

FIG. 16 illustrates the synthesis of PF-mOXDP, a representative polymer of the invention.

FIG. 17 illustrates the synthesis of PF-mCzP-mOXDP, a representative polymer of the invention.

FIG. 18 illustrates Ir-2R, an iridium-based phosphorescent emitter useful as a guest in films of the invention.

FIG. 19 illustrates Os-2, an osmium-based phosphorescent emitter useful as a guest in films of the invention.

FIG. 20 illustrates FIrpic, a blue emitter useful as a guest in films of the invention.

FIG. 21 graphically illustrates the UV-Vis spectra of two representative branched compounds of the invention.

FIG. 22 graphically illustrates the PL spectra of two representative branched compounds of the invention.

FIG. 23 illustrates the blue emitter compound FIr6, useful as a guest in representative films of the invention.

FIG. 24 illustrates the synthesis of MTP-CBP, a representative branched macromolecule of the invention.

FIG. 25 illustrates the synthesis of MTP-CF3CBP, a representative branched macromolecule of the invention.

### DETAILED DESCRIPTION OF THE INVENTION

In one aspect, the invention provides compounds used as hosts for phosphorescent emitters in electroluminescent devices. In one embodiment, the invention provides a polymer having a ground state to singlet excited state energy gap of from about 3.3 eV to about 3.5 eV and a triplet energy greater than about 2.6 eV. Compounds of the invention are designed as both emitters and as hosts for guest phosphorescent materials.

Compounds of the invention are useful as hosts for a broad range of phosphorescent emitters, from high-energy blue wavelengths to relatively low-energy red wavelengths. As hosts for high-energy phosphorescent emitters, the compounds of the invention have sufficiently high triplet energy states so as to facilitate high-energy transfer to guest phosphorescent emitters and/or prevent phosphorescence quenching. The triplet energy of a given material is less than the bandgap. To act as a proper host for triplet guest emitters, the compounds of the invention have a large first singlet excited state (S₁), meaning that the ground state to S₁ energy transition is greater than about 3.3 eV if the guest emitter is to have a sufficiently high-energy triplet state to emit light in the blue wavelength range. As used herein, the term “bandgap” refers to the energy transition between the ground state and the first singlet excited state (G→S₁). As emitters, the compounds will emit high-energy light in the blue to violet wavelength range of the visible spectrum. As used herein, the term “high-energy” refers to emission at a wavelength less than 420 nm. In one embodiment, the invention provides compounds having an emission wavelength maximum of from about 360 nm to about 420 nm.

In order to achieve the necessary large-bandgap requirement for hosting blue-emitters while maintaining a high level of processability, two broad classes of materials are disclosed in the present invention: polymers and branched compounds. Example 1 describes representative polymer and branched compounds of the invention.

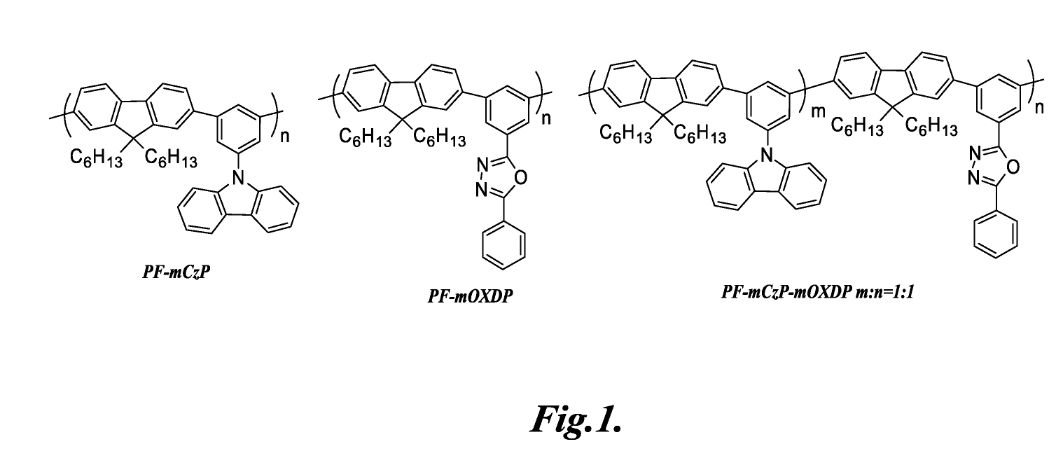

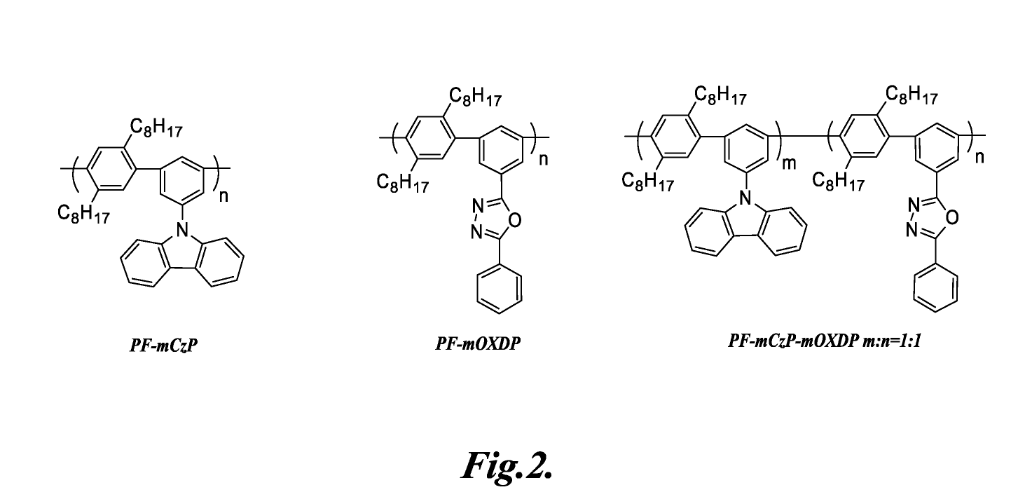

Polymer compounds of the invention achieve a high triplet-state through the use of meta-linkage in the conjugated backbone of the polymeric chain. An example of meta-linkage is illustrated in FIG. 1. The monomer units of all three compounds in FIG. 1 have a fluorene bonded in the para-position to a phenylene bonded in the meta-position. The meta-bonding of the phenylene reduces the bond and conjugation length in the backbone of the polymer. Reducing conjugation length results in a higher singlet energy state and, thus, the potential for a higher triplet energy state. By reducing the conjugation length between the two individual moieties in the backbone of the polymer (e.g., the fluorene and the phenylene illustrated in FIG. 1), the S₁ energy-state of the polymer compound is increased. FIG. 2 illustrates a further shortening of the conjugation length in the polymer chain using a second substituted phenylene unit instead of fluorene. The resulting materials from the phenylene-phenylene polymer chain have higher singlet energies than those of the phenylene-fluorene polymers illustrated in FIG. 1.

Compounds of the invention can be further modified by adding substituents to the polymer chain to control the electron- and hole-transporting properties of the material. Representative charge-transport substituents include electron-withdrawing oxadiazole groups and hole-donating carbazole groups. Hole-donating carbazole groups are illustrated in FIG. 1 (compound PF-mCzP), as well as FIG. 2 (compound PP-mCzP); electron-withdrawing oxadiazole groups are illustrated in FIG. 1 (compound PF-mOXDP), as well as FIG. 2 (compound PP-mOXDP). Both carbazole and oxadiazole groups can be incorporated into polymers of the invention, as illustrated in FIG. 1 (compound PF-mCzP-mOXDP), and FIG. 2 (compound PP-mCzP-mOXDP). By substituting both electron-withdrawing and hole-transporting groups onto the same polymer structure, the qualities of both groups will be manifested in the material (i.e., the polymer will be both hole-donating and electron-withdrawing). The purpose for adding either hole donating or electron withdrawing groups to the polymer backbone of the invention is to improve performance of electroluminescent (EL) devices fabricated with films incorporating the compounds of the invention. The addition of charge-transporting groups will tailor the conduction of holes and/or electrons through the films of the devices. By controlling the rate of travel of electrons and holes in an electroluminescent device, both the amount of electrons and holes that reach certain a specific region of a device can be controlled, as well as the speed at which they arrive at a particular location. By controlling the region in which electrons and holes recombine to form excited complexes that then emit light, the efficiency and general operability of the device can be optimized. Control of holes and electrons is advantageous because they should recombine in a material that is most efficiently excited by their recombination so as to produce the brightest light in the most efficient manner possible.

In addition to controlling the conjugation length and electron/hole-transporting properties of the polymers of the invention, the solubility, and thus the processability, of the polymers can be tailored by modifying the chemical structure. Processability is controlled in the invention by adding alkyl chains to at least one of the groups in the polymer backbone. Representative alkyl substitutions include nC₆H₁₃ (substituted onto fluorene, as illustrated in FIG. 1) and nC₈H₁₇ (substituted onto phenylene, as illustrated in FIG. 2). The addition of alkyl chains allow for higher solubility of the materials and, thus, a higher degree of processability. Higher solubility allows the materials to be deposited using solution-based techniques such as spin coating, drop coating, screen-printing, inject-printing, and other thin film techniques known to those skilled in the arts.

In one embodiment, the invention provides a polymer having the formula:

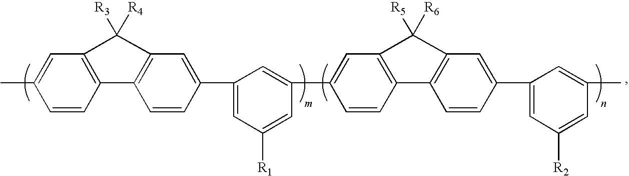

where R₁ and R₂ are independently selected from substituted and unsubstituted carbazole, thiophene, substituted and unsubstituted triphenyl amine, substituted and unsubstituted oxadiazole, substituted and unsubstituted triazine, substituted and unsubstituted benzothiadiazole, cyano, substituted and unsubstituted pyridine, substituted and unsubstituted quinoline, and substituted and unsubstituted quinoxaline; R₃, R₄, R₅, and R₆ are independently selected from branched and straight-chain alkyl groups having from one to twenty carbon atoms, or branched and straight-chain alkoxy groups having from one to twenty carbon atoms; m is an integer from 0 to about 60; n is an integer from 0 to about 60; and m+n≧1. The invention provides both homopolymers and copolymers. Homopolymers are provided when either m or n is zero. In a further embodiment, the invention provides a polymer, where m is zero. In a further embodiment, the invention provides a polymer, where n is zero. Non-zero values of both m and n will provide copolymers. The characteristics of copolymers can be altered by changing the ratio of m:n. Representative ratios of m:n include 1:1, 1:9, 1:4, 3:7, 2:3, 3:2, 7:3, 4:1, and 9:1. In a further embodiment, the invention provides a polymer, where the ratio of m:n is about 1:1.

In a further embodiment, the invention provides a polymer, where R₃, R₄, R₅, and R₆ are nC₆H₁₃.

In a further embodiment, the invention provides a polymer where n=0; R₁ is carbazole; and R₃, R₄, R₅, and R₆ are nC₆H₁₃.

In a further embodiment, the invention provides a polymer where m=0; R₂ is a phenyl-substituted oxadiazole; and R₃, R₄, R₅, and R₆ are nC₆H₁₃.

In a further embodiment, the invention provides a polymer where the ratio of m:n is about 1; R₁ is carbazole; R₂ is a phenyl-substituted oxadiazole; and R₃, R₄, R₅, and R₆ are nC₆H₁₃.

In one embodiment, the invention provides a polymer having the formula:

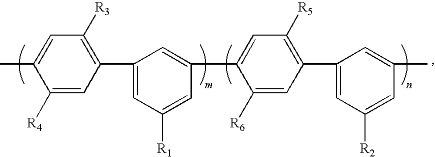

where R₁ and R₂, are independently selected from substituted and unsubstituted carbazole, substituted and unsubstituted thiophene, substituted and unsubstituted triphenyl amine, substituted and unsubstituted oxadiazole, substituted and unsubstituted triazine, substituted and unsubstituted benzothiadiazole, cyano, substituted and unsubstituted pyridine, substituted and unsubstituted quinoline, and substituted and unsubstituted quinoxaline; R₃, R₄, R₅, and R₆ are independently selected from branched and straight-chain alkyl groups having from one to twenty carbon atoms or branched and straight-chain alkoxy groups having from one to twenty carbon atoms; m is an integer from 0 to about 60; n is an integer from 0 to about 60; and m+n≧1. Representative ratios of m:n include 1:1, 1:9, 1:4, 3:7, 2:3, 3:2, 7:3, 4:1, and 9:1.

In a further embodiment, the invention provides a polymer, where m is 0.

In a further embodiment, the invention provides a polymer, where n is 0.

In a further embodiment, the invention provides a polymer, where the ratio of m:n is about 1:1.

In a further embodiment, the invention provides a polymer, where R₃, R₄, R₅, and R₆ are nC₈H₁₇.

In a further embodiment, the invention provides a polymer where n=0; R₁ is carbazole; and R₃, R₄, R₅, and R₆ are nC₈H₁₇.

In a further embodiment, the invention provides a polymer where m=0; R₂ is a phenyl-substituted oxadiazole; and R₃, R₄, R₅, and R₆ are nC₈H₁₇.

In a further embodiment, the invention provides a polymer where the ratio of m:n is about 1; R₁ is carbazole; R₂ is a phenyl-substituted oxadiazole; and R₃, R₄, R₅, and R₆ are nC₈H₁₇.

In one embodiment, the invention provides branched compounds having the formula:

(E-L)ₙX

where n is 0, 1, 2, 3, or 4; X is an alkyl, heteroalkyl, or aryl core that is linked to charge-transporting moiety E by linker L; and E independently at any occurrence is the same or different from any other E in the compound.

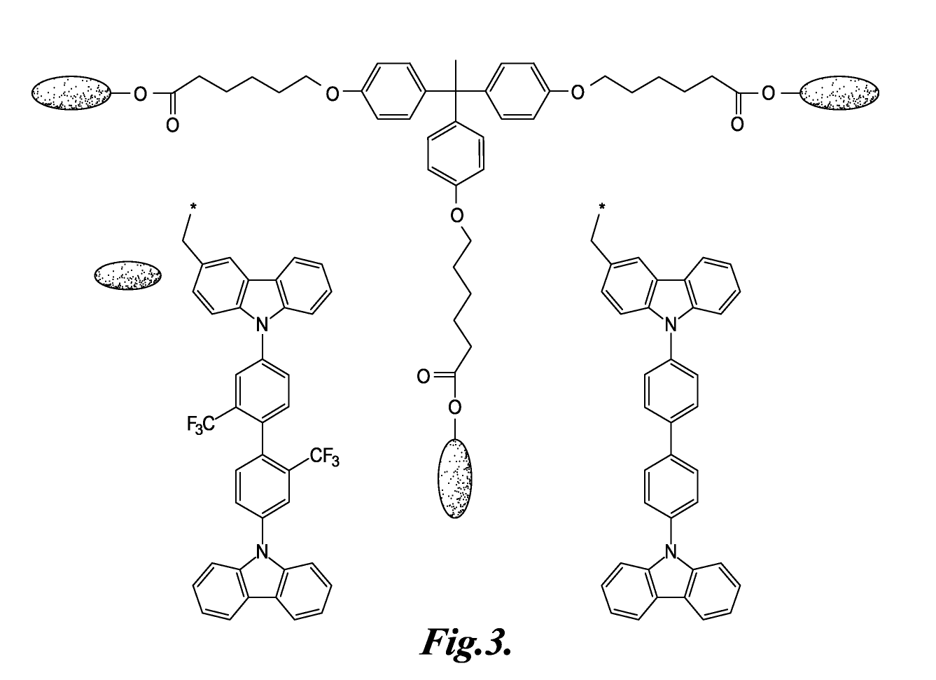

Branched molecules are also useful in making compounds of the invention. As with polymers of the invention, branched compounds of the invention have a high triplet energy level so as to facilitate energy transfer to high-energy phosphorescent emitter guest compounds and/or to avoid host quenching of high-energy phosphorescent emission from guest compounds. Representative branched compounds of the invention are illustrated in FIG. 3, and their syntheses are described in Example 1. The approach taken to maximizing the triplet energy level of branched compounds is to electronically isolate charge-transporting moieties by introducing an insulating core.

Branched compounds of the invention have three parts: a core, two or more linkers, and two or more charge-transporting moieties. The core is an atom or group of atoms to which two or more linkers are covalently coupled. In one embodiment, the core is an alkyl, heteroalkyl, or aryl group having two or more branches (e.g., 2, 3, or 4) diverging from its central atom or group of atoms. The linker is an atom or group of atoms that covalently link the core to the charge-transporting moiety. In one embodiment, the linker is an alkyl or heteroalkyl group. Representative linkers include ethers and esters. The charge-transporting moieties of the compound are versions of high-triplet-energy small-molecule materials modified for attachment to a core via a linker. Several charge-transporting moieties can be attached to the core, yielding a number of charge-transporting moieties in a single branched compound structure. All of the arms of the branched compound need not be substituted with charge-transporting moieties. Different arms of the material can be substituted with hole- or electron-transporting moieties or nonfunctional moieties that are designed to shape the overall physical profile of the molecule and/or the way that the molecule interacts with adjacent molecules.

In a further embodiment, the invention provides a compound, where E has an emission wavelength maximum of from about 360 nm to about 420 nm.

In a further embodiment, the invention provides a compound, where L independently at each occurrence is at least one of an alkyl, heteroalkyl, or aryl group.

In one embodiment, the invention provides a compound having the formula:

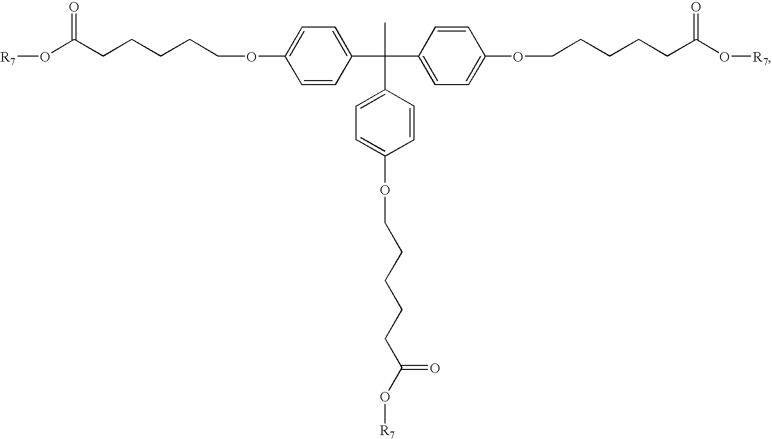

where R₇ is selected from the group:

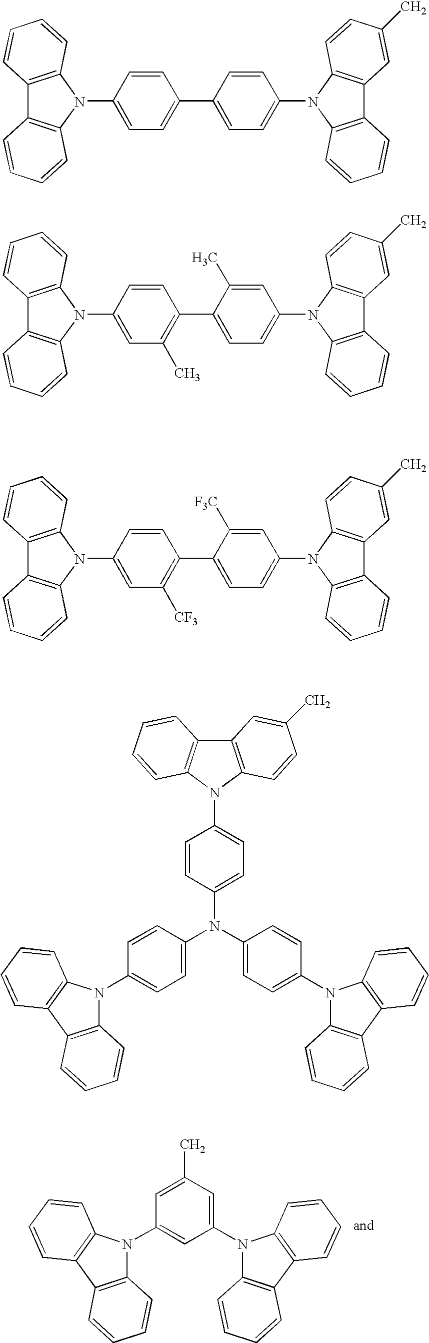

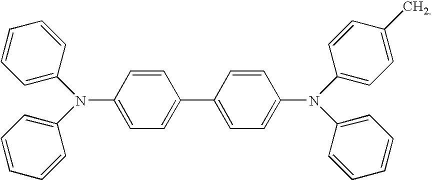

In the above compound, the core can be considered to be the 1,1,1-tris(phenoxy) ethane moiety, and the linker can be considered to be the hexanoic acid moiety.

For the compounds of the invention to effectively host phosphorescent guest emitters, a pathway for excitation of the guest emitter through the host exists and the host does not substantially quench the phosphorescence of the guest emitter.

Excitation can be facilitated in two different ways: energy transfer and charge-trapping. Energy transfer can occur by Förster (long-range, dipole induced) and/or Dexter (short-range, electron tunneling) energy transfer from the host to the guest. Alternatively, direct, sequential trapping of both electrons and holes on the guest (“charge trapping”) can provide excitation energy to the guest phosphorescent emitter. In the energy transfer process, the host compound is excited either by light or by electricity, photoluminescence (PL) or electroluminescence (EL). When the host material is excited, the singlet (S₁) state is populated. If the host material has a triplet state available, the triplet state may become populated via intersystem crossing. From the excited singlet state of the host material, energy transfer can occur between the singlet state of the host material and a singlet state of a guest phosphorescent emitter. An excited singlet state in the guest can populate a triplet state via intersystem crossing. Phosphorescence of the guest can occur if a triplet state is populated. The energy level of the triplet state of the guest emitter will determine the wavelength of light of emitted. For blue emission from a phosphorescent guest, the triplet state of the guest emitter will need to be relatively high (below 500 nm), and in order to populate the high-energy triplet state of the guest phosphorescent emitter, the host compound has an equally high or higher energy bandgap. High-energy phosphorescent emission is in the range of 400-500 nm and the corresponding triplet energy of emission is from about 2.6 eV to 3.2 eV.

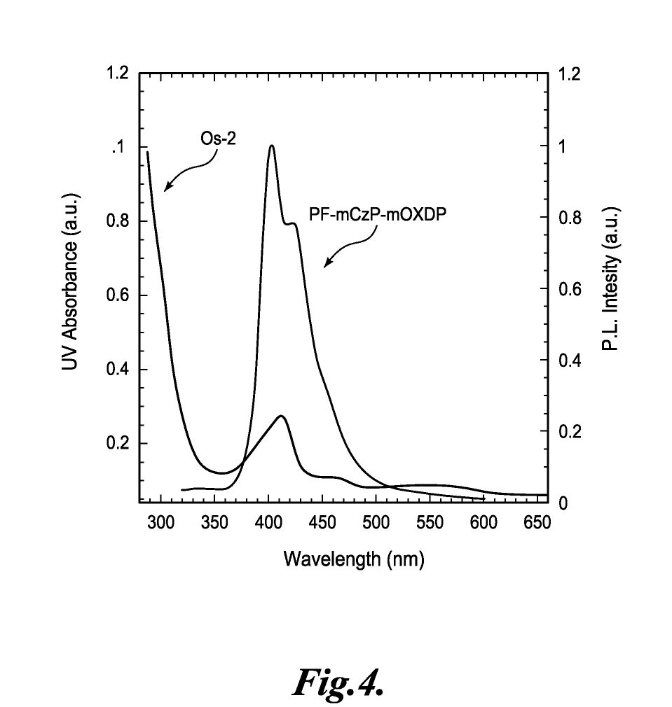

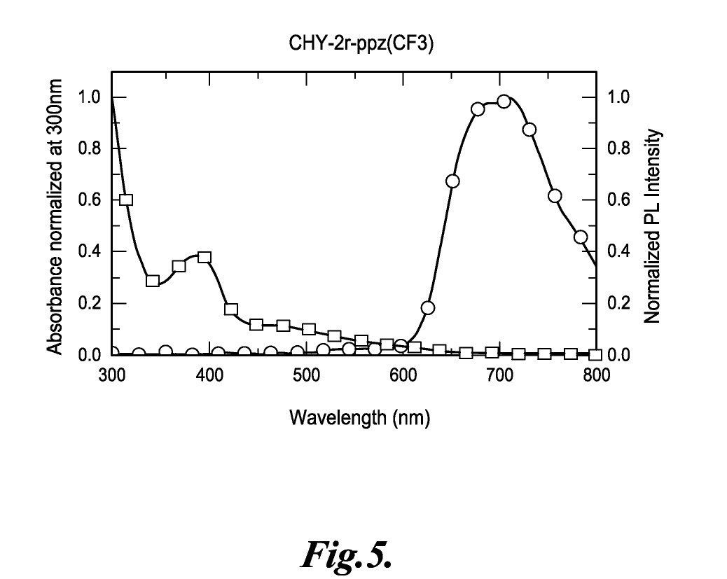

Energy transfer between host and guest can be characterized using the photoluminescent spectrum of the host material and the absorbance spectra of the guest material. If the photoluminescence of the host has any wavelengths overlapping the absorption of the guest, then energy may be transferred between the two materials. The amount of energy that is transferred is relative to the size of the overlap between the host emission and guest absorption. An indication of the size of this energy overlap is the area of the spectral region shared between the emission of the host and absorption of the guest. An example of strong overlap between emission and absorption is illustrated in FIG. 4, where the emission of PF-mCzP-mOXDP is strongly overlapping the absorption band of the osmium guest emitter complex. Weaker, but still effective, overlap between emission and absorption is illustrated in FIG. 5, where, although the emission of the host only tails-off in the region where the guest emitter begins to absorb, there is still effective transfer of energy between the two.

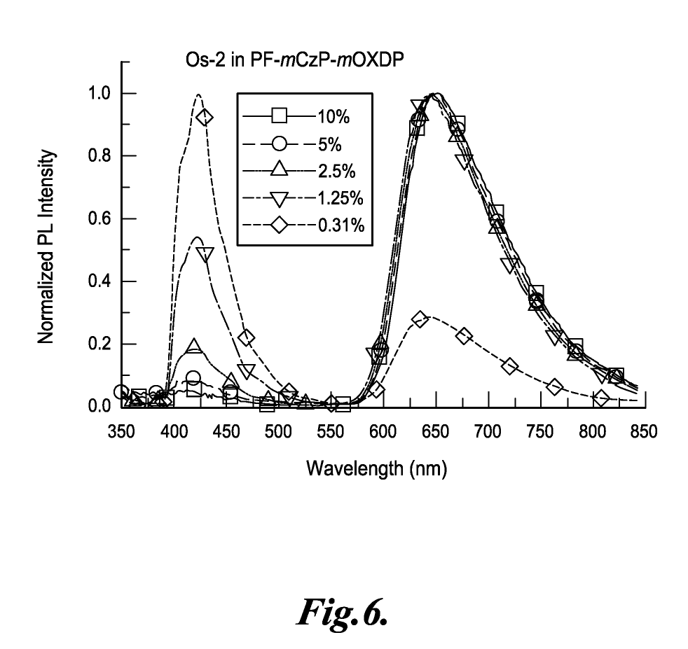

The amount of energy transferred between the host and guest materials will be dictated not only by the overlap of the wavelengths of the emission region of the host and the absorption region of the guest, but will also be determined by the relative amounts of the guest in the host material. The effect of guest concentration on the photoluminescence spectra of a guest-host film is illustrated in FIG. 6, where the phosphorescent red emitter Os-2 is a dopant in the host compound of the invention PF-mCzP-mOXDP. The PF-mCzP-mOXDP host material emits in the blue region, and the guest material emits in the red region. As illustrated in FIG. 6, as the amount of emitter guest in the host material increases, the peak in the blue region slowly decreases and the peak in the red region increases in size. As the peaks change size, energy is transferred by the host emitter to the guest phosphorescent emitter emitting in the red wavelength region. As the concentration of the guest emitter increases, increased emissive energy from the host material is transferred to the guest material, thus decreasing the size of the peak of the emitter host in the blue region and increasing the size of the emitter guest in the red region. FIG. 6 is also illustrative of another important facet of the invention: the energy of the triplet energy state of the host and guest materials. The energy of the triplet state of the guest material defines the wavelength at which the guest emits. If the triplet energy of the host material is lower than the triplet energy emissive state of the guest material, phosphorescence quenching of the emission of the guest material would occur because of the lower triplet energy state of the host material. The triplet energy of the guest would be transferred back to the host instead of releasing the energy via phosphorescent emission. Thus, if emission from a phosphorescent guest is observed, it can be positively stated that the energy of the triplet energy level of the host is higher than that of the guest.

Because of the energetic requirements for phosphorescent emission via excitation from a photoluminescent host material (i.e., because the host material must have a higher triplet energy state than the guest material triplet level energy state), the issue of phosphorescence quenching will not likely arise in a purely photoluminescent situation. However, phosphorescence quenching is a concern when dealing with electroluminescence (e.g., in an electroluminescent device of the invention) because the host material may be excited by charge trapping instead of photoluminescence. When a guest emitter is excited by charge trapping and forms an excited triplet state, it can decay via an emissive phosphorescent route. However, if the triplet energy level of the host material is lower than the triplet energy level of the guest emitter material, phosphorescence quenching may occur and reduce (or eliminate) the emission from the electroluminescent device.

The second mechanism by which the guest emitter molecules can become excited and phosphoresce is charge trapping. Charge trapping uses the host material as an inert medium for transmitting holes and electrons from an anode and a cathode of an electroluminescent device into an emitter guest material. The guest emitter is excited by the recombined electrons and holes and facilitates phosphoresces via electronic excitation, as opposed to the absorption of energy from the host. Charge trapping allows direct exciton formation on the guest phosphorescent material, eliminating the need to excite the host, and allowing for improved carrier collection, exciton formation, and recombination in the guest. One characteristic of a charge trapping system for phosphorescent emission from a guest material is that the host material should not quench the phosphorescence of the guest. As discussed above, the requirements for a host material in a charge trapping system include a high-energy triplet state. The energy of the triplet state should be greater than the energy of the triplet state of the guest material so as to block any energy transfer from guest to the host. Any transfer of energy between guest and host will diminish the amount of energy that is transferred into phosphorescence, resulting in diminution of the brightness of any device made using this system, as well as diminishing the device's efficiency.

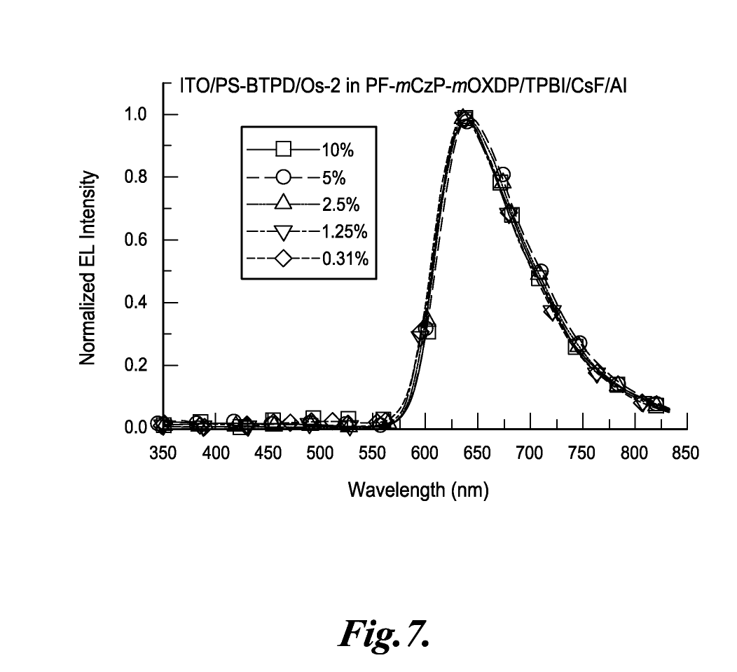

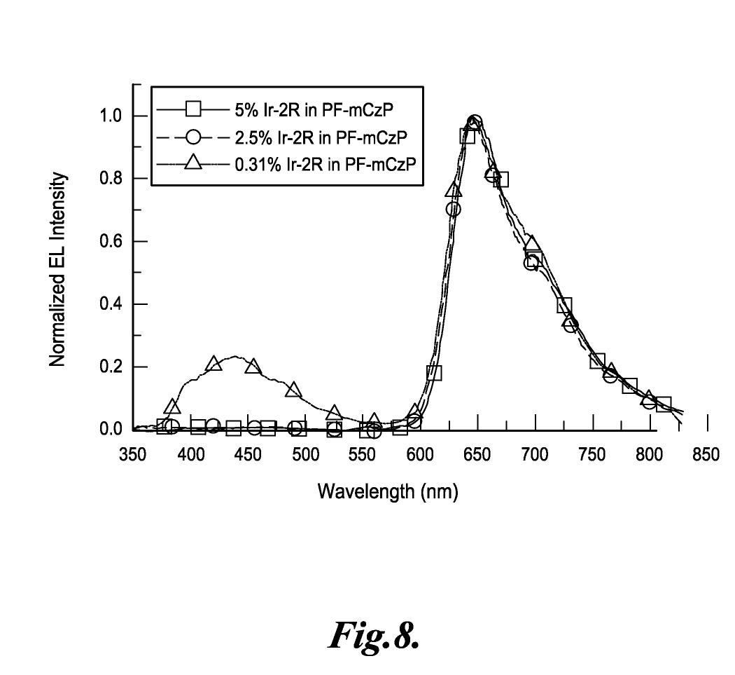

An example of a charge trapping electroluminescent device is shown in FIG. 7. The exemplary red device shows only emission from the guest emitter Os-2 in the 600-800 nm wavelength range. The host material, PF-mCzP-mOXDP, emits in the blue-violet region of the spectrum and is not present in the electroluminescent data. The difference between the EL (FIG. 7) and PL (FIG. 6) spectra of similar films of Os-2/PF-mCzP-mOXDP indicates the dominant role of the charge trapping and recombination in the EL process. The HOMO and LUMO energy levels of Os-2 are −5.0 eV and −2.7 eV (respectively) and the HOMO and LUMO of PF-mCzP-mOXDP are −5.7 eV and −2.4 eV (respectively), as determined by the cyclic voltammetry. As a result of the energy levels of the guest and host, the Os-2 complex functions as both a hole and electron trap. Thus, the main function of the PF-mCzP-mOXDP host in the device is to transport injected charges efficiently to the Os-2 trapping sites. Charge trapping sites are dispersed within the entire EL layer. The Os-based emitter characterized by EL in FIG. 7 can be compared to FIG. 8, the electroluminescence spectra of an Ir-based red emitter and a PF-mCzP host (device structure: ITO/PS-BTPD-PFCB/Ir-2 (guest)-PF-mCzP (host) film/TPBI/CsF/Al; the synthesis of the hole-transporting material PS-BTPD-PFCB is described in Example 3). Peaks for both the host and the emitter are detected in the electroluminescence spectra of FIG. 8, while only the single guest emitter peak is detected in FIG. 7. The difference between spectra of the figures shows that there are both types of excitation (charge trapping and energy transfer) occurring in the device used to produce the EL spectra illustrated in FIG. 8. For EL devices, it is important to note that the host material has higher triplet energy than that of the guest emitter. While not always functioning as an emitter and source of energy transfer to the guest emitter, the properties of the host are related to device efficiency. If the triplet energy state of the host material were lower than the triplet energy state that gives rise to the electroluminescent emission peak, then phosphorescence quenching would occur and no electroluminescence would be generated.

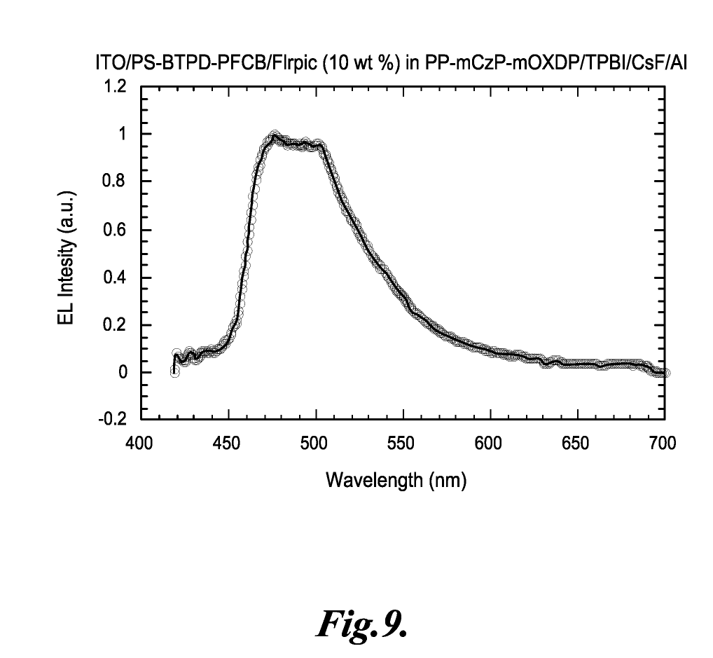

Representative compounds of the invention support high-energy phosphorescent emitter guest materials. FIG. 9 illustrates an EL spectrum from a device incorporating the blue guest emitter FIrpic in a representative host material of the invention, PP-mCzP-mOXDP. The electroluminescent spectrum shows only emission from the high-energy blue guest material, indicating that the triplet energy level of PP-mCzP-mOXDP is greater than or equal to the triplet energy level of FIrpic (about 2.6 eV).

The numeric value (in eV or nm) of the triplet energy state is difficult to quantify because it requires low temperature testing and elaborate analytical equipment. However, it can determined that if emission is detected in a guest-host system where the guest emitter is a triplet emitter, then the triplet energy of the host material will be equal to, or higher than, the triplet energy of the phosphorescent triplet emission band of the guest material.

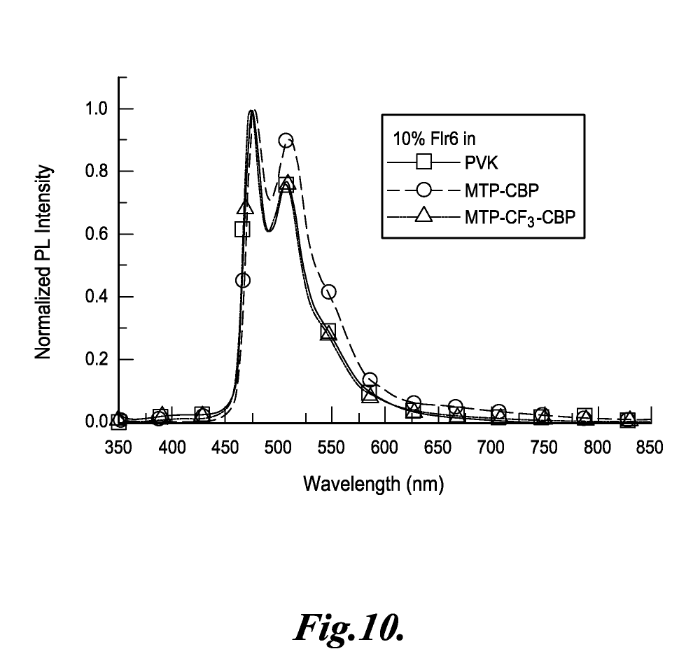

Branched compounds of the invention are also capable of supporting both energy transfer to a guest and charge-trapping for electroluminescent operation. FIG. 10 graphically illustrates spectra from representative electroluminescent devices incorporating branched molecules of the invention. The branched molecules have a high-energy triplet state, as determined by the electroluminescence of the phosphorescent blue triplet emitter FIr6. The energy of the triplet host material can be tailored by altering the groups attached to the charge-transporting moieties of the branched material. For example, by introducing two methyl group onto the biphenyl moiety of 4,4′-bis(9-carbazolyl)-biphenyl, which has the triplet energy level around 2.6 eV, the triplet energy level of the 4,4′-bis(9-carbazolyl)-2,2′-dimethyl-biphenyl can be increased to 3.0 eV.

In another aspect, the invention provides a film that includes a compound of the invention and a phosphorescent emitter. In addition to compounds, the invention also provides for the use of those compounds integrated into films with an emissive guest material (“guest-host”). In guest-host systems, the host material typically provides a benefit to the guest material, or vice versa. In films of the present invention, the host material provides either energy transfer to the guest material or the host material acts as a passive matrix and provides a pathway for charge transport/charge trapping. Guest-host films of the invention can be prepared by a solution route where both the guest compound and the host compound are dissolved in a solvent. Representative films of the invention incorporate phosphorescent guest compounds in the host at about 0.1%-20% (by weight). The solvated solution of both guest and host material can then be used to form a film by any number of film-forming processes known to those skilled in the art. These solution-based film-forming processes include spin-coating and drop-coating. Films of the invention are typically formed on a substrate. The substrate can be a component of an electroluminescent device (e.g., an OLED).

In one embodiment, the invention provides a film, where the phosphorescent compound has an emission wavelength maximum of from about 400 nm to about 700 nm.

In one embodiment, the invention provides a film, where the compound has an emission wavelength range that overlaps with the absorption wavelength range of the first phosphorescent compound.

In one embodiment, the invention provides a film, where the compound has a triplet energy greater than the triplet energy of the first phosphorescent compound.

In one embodiment, the invention provides a film, where the compound's triplet energy is sufficiently greater than the phosphorescent compound's triplet energy that there is no return energy transfer to the host compound from the phosphorescent compound.

In one embodiment, the invention provides a film, where the film further includes a second phosphorescent compound.

In another aspect, the invention provides an electroluminescent device, including a first electrode, a second electrode, and a film intermediate the first and second electrodes that includes a compound of the invention and a phosphorescent emitter.

Films that incorporate compounds of the invention can further be incorporated into electroluminescent devices. Electroluminescent devices are described for specific compounds of the invention in Example 1 and discussed generally in Example 2. The most common electroluminescent device is the organic light-emitting diode (OLED). The simplest structure for an OLED is a three-component structure consisting of an emissive film intermediate two electrodes. One electrode is an anode, the other electrode is a cathode. The electrodes inject holes and electrons, and the charged species recombine in the emissive film to form an exciton and emit light at a wavelength characteristic of the excited-state energy level of the emissive material in the film. Compounds of the invention are electroluminescent and thus able to be excited in an OLED structure and emit light at a wavelength that is in the blue or violet region of the visible spectrum. Films of the present invention incorporate compounds of the invention as well as phosphorescent emitters known to those skilled in the arts. Representative phosphorescent emitters include Os-2, Ir-2R, FIrpic, FIr6, and Ir(ppy)₃.

Electroluminescent devices of the invention can operate by way of two different mechanisms that allow the triplet energy state of the guest phosphorescent emitter to be excited and emit light. The first mechanism is energy transfer. Energy transfer is a mechanism that uses the host material as an active component in the electroluminescence of the entire device. In energy transfer electroluminescent devices, the host material is excited and emits at a blue or violet wavelength. The guest material is excited in its singlet state via energy transfer from the singlet state of the host material. The large spin-orbit coupling for heavy-metal guests leads to efficient intersystem crossing from the singlet excited state to the triplet state, and thereby enables phosphorescence from the triplet state. The wavelength of light emitted from the guest phosphorescent material will be determined by the energy of the excited triplet state of the material.

The second mechanism by which electroluminescence is generated in devices of the invention is charge trapping. In the charge-trapping mechanism, holes and electrons are generated at the electrodes of the device and recombine in the film of the invention at recombination sites on the phosphorescent guest materials. In charge-trapping devices of the invention, the host compounds of the invention act as charge-transporting matrices for emissive phosphorescent guest materials. In electroluminescent devices of the invention, it remains important that the triplet energy level of the host material is higher than the triplet energy level of the emissive material so as to avoid phosphorescence quenching (i.e., rendering of the device non-luminescent).

Electroluminescent devices of the invention may also incorporate either hole- or electron-transporting materials, or both, into the overall device structure. These charge-transporting materials allow for both efficient injection of charge from the electrodes into the recombination zone (located in the films of the invention) and also allow for tuning of the number and location of holes and/or electrons in the device. In addition, the hole-transporting layer can also function as an electron-blocking and exciton-confining layer at the anode side, and the electron-transporting layer can function as a hole-blocking and exciton-confining layer at the cathode side.

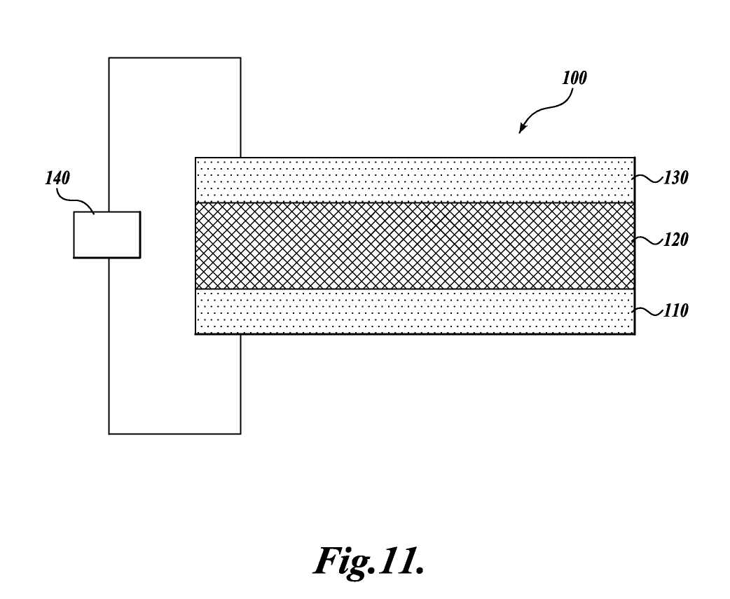

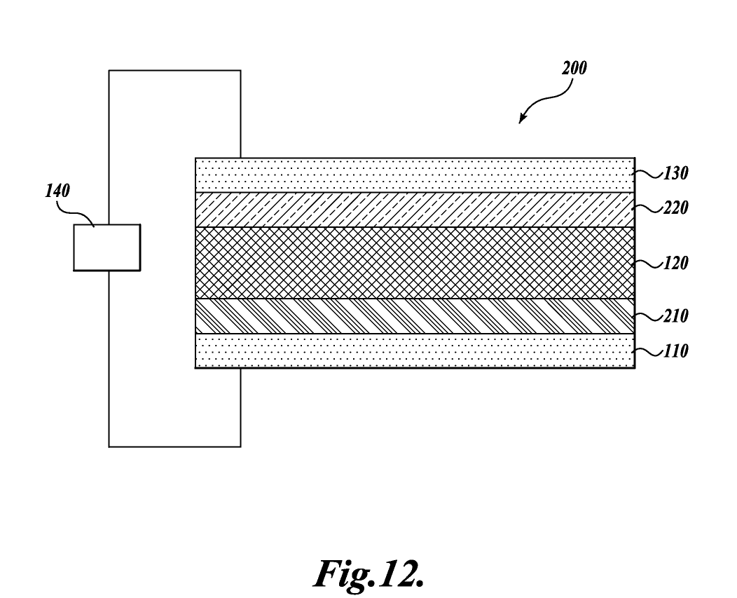

Electroluminescent devices of the invention can be fabricated using well known microelectronic and semiconductor processing techniques known to those skilled in the arts. A typical device structure 100 is illustrated in FIG. 11 and will include a first electrode 110, typically a transparent electrode such as indium tin oxide (ITO) deposited on a substrate. On top of the first electrode, film-forming materials in liquid form are deposited, typically by spin coating, drop coating, or another solution-based deposition technique. The film deposition technique forms a solid film that can then be cured at an elevated temperature so as to evaporate any remaining solvent. The final product is a solid film of the invention 120 containing both a host material of the invention and a guest phosphorescent emitter material. On top of the film of the invention, a second electrode 130 is typically deposited. A representative second electrode is a metallic electrode deposited by an evaporation or sputtering technique. Typical second electrode materials include gold, silver, aluminum, magnesium, calcium, CsF, LiF, Ca, combinations of the materials (i.e., aluminum-capped CsF), and other electrode materials known to those skilled in the art. For more complex devices 200, as illustrated in FIG. 12, a hole-injection layer 210 and an electron injection layer 220 can optionally be incorporated into the device to improve charge injection and transport. In the representative devices described above, the first electrode 110 will act as an anode and will produce holes in the device. To improve the efficiency of hole injection into the device, a hole injection 210 layer may be deposited on the first electrode before the film of the invention is formed. A hole-injection layer can be deposited either by a solution-based or vapor-based technique. Once the hole-transporting layer is deposited, the film of the invention 120 can then be formed on top of the hole-transporting layer. When the film is cured and solidified, an electron-transporting 220 layer can optionally be deposited upon the film of the invention. Deposition of the electron-transporting layer can be done using a solution-based or vapor-based technique. Finally, on top of the electron-transporting layer, the second electrode 130 (cathode) material can be deposited, typically using an evaporative technique. The completed device can be operated by attaching the anode and cathode to an electrical power supply 140. When the device is run in forward bias, the electrons and holes produced at the cathode and anode, respectively, will migrate through any charge-transporting layers and will recombine in the film of the invention. Recombination will either excite the host emissive material that would in turn transfer energy to the guest phosphorescent emissive material allowing it to phosphoresce; or the host material would act as a charge-transport layer, allowing the holes and electrons to recombine directly on the phosphorescent compounds, creating a local exciton and phosphorescence.

When host compounds of the invention are used in electroluminescent devices, an additional consideration arises: the level of the HOMO and LUMO levels of the host material. The host should possess suitable HOMO and LUMO energy levels to facilitate charge injection and transport. The HOMO level of the compound should be near the same energy as the work function of the anode or hole-injection layer, if present. The LUMO should be about the same energy as the work function of the cathode or electron-injection layer, if present.

In one embodiment, the invention provides a device, where the film further comprises a second phosphorescent compound.

In one embodiment, the invention provides a device further including an electron-transport material intermediate the film and the first electrode.

In one embodiment, the invention provides a device further including a hole-transporting material intermediate the film and the second electrode.

The following examples are provided for the purpose of illustrating, not limiting, the invention.

### EXAMPLES

### Example 1

### Synthesis and Characterization of Representative Compounds of the Invention

### Conjugated Polymers of the Invention Having Meta-Linkage

The structures of PF-mCzP, PF-mOXDP and PF-mCzP-mOXDP are illustrated in FIG. 1. The structures of PP-mCzp, PP-mOXDP, and PP-mCzp-mOXDP are illustrated in FIG. 2. The alternating copolymers PF-mCzP and PF-mOXDP were synthesized by the Suzuki coupling reaction between fluorene diboronate and 9-(3,5-dibromophenyl)-9H-carbazole, 2-(3,5-dibromophenyl)-5-phenyl-1,3,4-oxadiazole, respectively. A bipolar, random copolymer, PF-mCzP-mOXDP containing both the electron-transporting oxadiazole- and the hole-transporting carbazole-phenylene was also synthesized for balanced charge injection and transport. The structures of the polymers were confirmed by ¹H NMR.

All synthesized polymers are readily soluble in common organic solvents, including toluene, chloroform, and THF. The molecular weight of the synthesized polymers was determined by gel permeation chromatography (GPC) using THF as the eluent and calibrating against a polystyrene standard. The results are summarized in Table 1. The weight-average molecular weights (M_{W}) of these polymers ranged from 18,000 to 28,300 with a typical polydispersity less than 2.0.

TABLE 1
Molecular weights and thermal properties of polymers of the invention.

| Polymer | Mₙ | M_{w} | DSC T_{g} (° C.) | TGA 5% (° C.) |
|---|---|---|---|---|
| PF-mCzP | 14,200 | 26,500 | 190 | 429 |
| PF-mOXDP | 9,200 | 18,800 | 173 | 412 |
| PF-mCzP-mOXDP | 16,600 | 28,300 | 190 | 408 |

The thermal properties of these copolymers were investigated by thermogravimetric analysis (TGA) and differential scanning calorimetry (DSC). The data are listed in Table 1. All polymers showed excellent thermal stability and have <5% weight-loss at temperatures beyond 400° C. A distinct glass transition temperature (T_{g}) of 173-190° C. was observed for the three polymers in Table 1. These glass-transition temperatures are much higher than a simple fluorene homopolymer (T_{g}˜75° C.). These high T_{g} values are attributed to the rigid carbazole-phenylene and oxadiazole-phenylene moieties that significantly enhance the chain rigidity and restrict the segment motion. Employing these high T_{g} polymers as hosts in light-emitting devices (e.g., OLEDs) will significantly increase device stability and prolong device lifetime.

The electrochemical behavior of the polymers was investigated by cyclic voltammetry (CV). In CV measurements, no reduction waves could be observed for all the polymers. Only PF-mCzP exhibits a quasi-reversible oxidation wave and its HOMO level is calculated to be −5.6 eV. The introduction of an electron-deficient oxadiazole-containing phenylene group in PF-mOXDP results in a CV-plot characteristic of irreversible oxidation, and an increased ionization potential. Generally, the ionization potential of the polymer increases with increasing oxadiazole content. The HOMO energy level is −5.7 eV for PF-mCzP-mOXDP and −5.9 eV for PF-mOXDP.

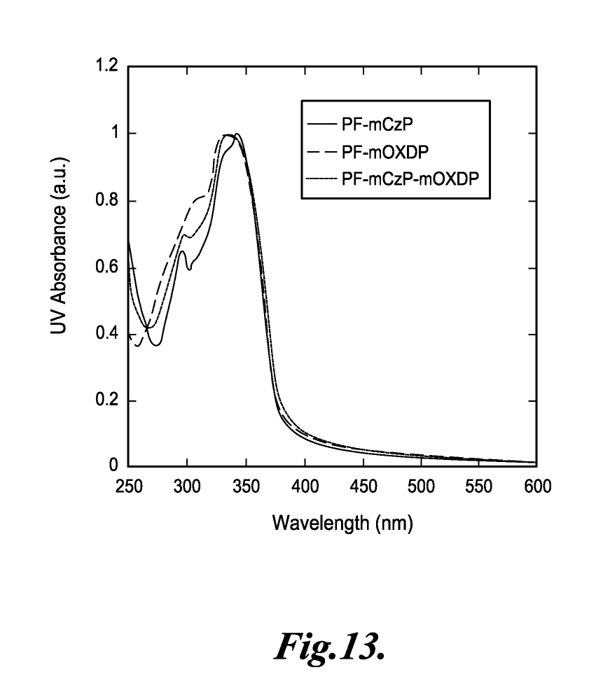

FIG. 13 shows the UV-Vis absorption spectra of PF-mCzP, PF-mOXDP, and PF-mCzP-mOXDP. The absorption spectra of PF-mCzP and PF-mCzP-mOXDP are similar. Both show an absorption λₘₐₓ at 342 nm and with a side peak at 296 nm. The absorption of PF-mOXDP is blue-shifted, with a λₘₐₓ at 336 nm and a side-peak at 309 nm. The main-peaks can be assigned to the delocalized π-π* electron transitions along the conjugated polymer backbone, while the side-peaks result from the electronic transitions of the monomer repeating units. The onset of the absorption of PF-mCzP, PF-mOXDP, and PF-mCzP-mOXDP is at 379 nm, corresponding to a band gap of 3.3 eV.

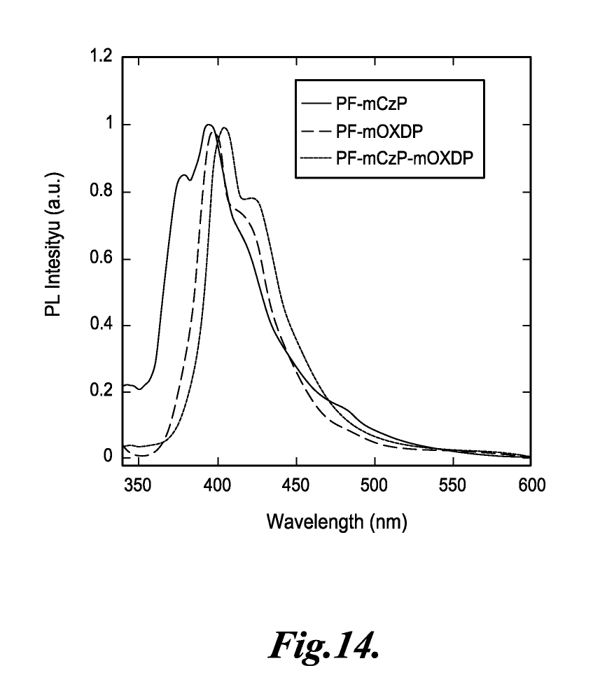

The photoluminescence (PL) spectra of PF-mCzP, PF-mOXDP, and PF-mCzP-mOXDP are graphically illustrated in FIG. 14. All polymers emit in the purple-blue region of the visible spectrum. The high-energy emission in the purple-blue wavelengths indicates that the introduction of a meta-phenylene linkage into the polymer backbone effectively interrupts the conjugation and increases the band gap.

The performance characteristics of OLED devices made using the PF host compounds of the invention as the only component of the emissive layer are shown in Table 2. The device structure is ITO/PEDOT:PSS/Emissive Layer/CsF/Al.

TABLE 2
Performance of representative devices of the invention.

| | Vₒₙ | ηₘₐₓ | Bₘₐₓ | LE | CIE coordinates | |
|---|---|---|---|---|---|---|
| Emissive Layer | (V) | (%) | (cd/m²) | (cd/A) | x | y |
| PF-mCzP | 5.5 | 0.35 | 529 | 0.24 | 0.18 | 0.11 |
| PF-mOXDP | 4.5 | 0.07 | 39 | 0.015 | 0.17 | 0.07 |
| PF-mCzP-mOXDP | 5 | 0.52 | 349 | 0.11 | 0.17 | 0.05 |

Vₒₙ: Turn-on voltage
ηₘₐₓ: Maximum external quantum efficiency
Bₘₐₓ: Maximum brightness
LE: Luminous efficiency

All three polymers in Table 2 show emission in the UV-blue region. By introducing electron-transporting (oxadiazole) and hole-transporting (carbazole) moieties into the polymer backbone (PF-mCzP-mOXDP), device performance (notably external quantum efficiency) is improved via balanced charge injection and transport.

Improved performance of the device-structure used in Table 2 can be achieved by inserting a hole transporting/electron-blocking layer intermediate the PEDOT:PSS hole-injection layer and the emissive layer. When PVK is used as a hole-transport layer in a device incorporating PF-mCzP, the enhanced external quantum efficiency rises to 2.31% compared to an external efficiency of 0.35% without the hole-transport layer. Other attributes of the PVK-enhanced device are: Vₒₙ=5.6 V; Vₘₐₓ=1930 cd/m²; and LE—0.88 cd/A.

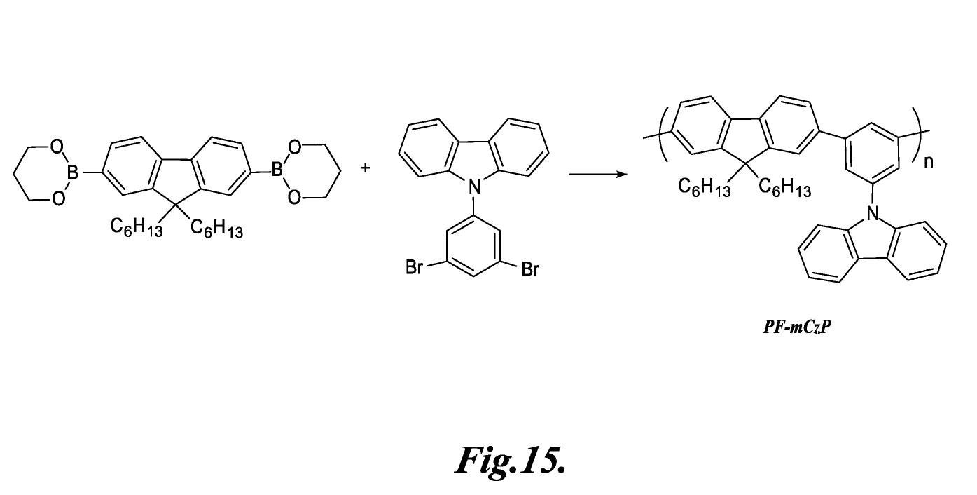

PF-mCzP. The synthesis of PF-mCzP is schematically illustrated in FIG. 15. To a solution of 9,9-dihexylfluorene-2,7-bis(trimethylene boronate) (502 mg, 1 mmol) and 9-(3,5-dibromophenyl)-9H-carbazole (401 mg, 1 mmol) in toluene (10 mL) were added aqueous potassium carbonate (2 M, 1.65 mL) and aliquate 336 (20 mg). The above solution was degassed, and tetrakis(triphenylphosphine)palladium (5 mg) was added in one portion under a nitrogen atmosphere. The solution was refluxed under nitrogen for 3 days. The polymerization was end-capped with phenylboronic acid for 6 h, followed by bromobenzene for another 6 h. After this period, the mixture was cooled and poured into a mixture of methanol and water. The crude polymer was filtered, washed with excess methanol, and dried. The crude polymer was further purified by redissolving the polymer into THF and reprecipitating in methanol several times to give PF-mCzP (380 mg, 63%). ¹H NMR (300 MHz, CDCl₃): δ¹H NMR. (300 MHz, CDCl₃): δ 8.21 (t, 2H, J=7.8 Hz), 8.07 (s, 1H), 7.90-7.77 (m, 6H), 7.70 (s, 2H), 7.61 (d, 2H, J=8.1 Hz), 7.49 (t, 2H, J=7.2 Hz), 7.36 (t, 2H, J=7.5 Hz), 2.05 (bs, 4H), 1.06 (bs, 16H), 0.71 (t, 6H, J=6.9 Hz).

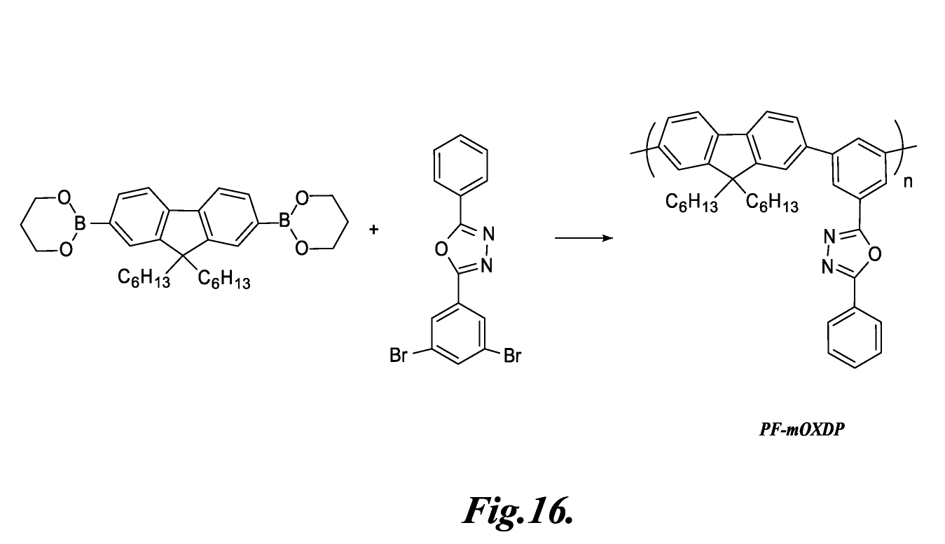

PF-mOXDP. The synthesis of PF-mOXDP is schematically illustrated in FIG. 16. To a solution of 9,9-dihexylfluorene-2,7-bis(trimethylene boronate) (251 mg, 0.5 mmol) and 2(3,5-dibromophenyl)-5-phenyl-1,3,4-oxadiazole (190 mg, 0.5 mmol) in toluene (10 mL) were added aqueous potassium carbonate (2.0 M, 0.8 mL) and aliquate 336 (10 mg). The above solution was degassed, and tetrakis(triphenylphosphine)palladium (2.5 mg) was added in one portion under a nitrogen atmosphere. The solution was refluxed under nitrogen for 3 days. The polymerization was end-capped with phenylboronic acid for 6 h, followed by bromobenzene for another 6 h. After this period, the mixture was cooled and poured into a mixture of methanol and water. The crude polymer was filtered, washed with excess methanol, and dried. The crude polymer was further purified by redissolving the polymer into THF and reprecipitating in methanol several times to give PF-mOXDP (431 mg, 38%). ¹H NMR (300 MHz, CDCl₃): δ ¹H NMR (300 MHz, CDCl₃): δ 8.45 (s, 2H), 8.24-8.21 (m, 2H), 8.15 (s, 1H), 7.94 (d, 2H, J=8.1 Hz), 7.8 (d, 2H, J=8.1 Hz), 7.70 (s, 2H), 7.56 (s, 3H), 2.17 (bs, 4H), 1.14 (bs, 16H), 0.78 (t, 6H, J=7.2 Hz).

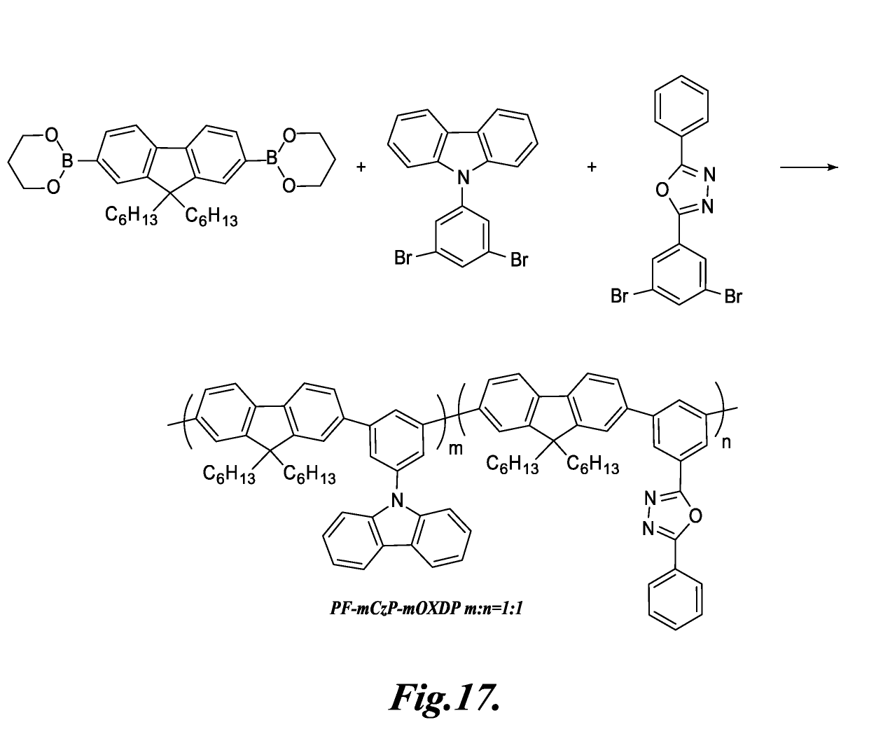

PF-mCzP-mOXDP. The synthesis of PF-mCzP-mOXDP is schematically illustrated in FIG. 17. To a solution of 9,9-dihexylfluorene-2,7-bis(trimethylene boronate) (502 mg, 1 mmol), 9-(3,5-dibromophenyl)-9H-carbazole (200 mg, 0.5 mmol), and 2(3,5-dibromophenyl)-5-phenyl-1,3,4-oxadiazole (190 mg, 0.5 mmol) in toluene (10 mL) were added aqueous potassium carbonate (2.0 M, 1.65 mL) and aliquate 336 (20 mg). The above solution was degassed, and tetrakis(triphenylphosphine)palladium (5 mg) was added in one portion under a nitrogen atmosphere. The solution was refluxed under nitrogen for 3 days. The polymerization was end-capped with phenylboronic acid for 6 h, followed by bromobenzene for another 6 h. After this period, the mixture was cooled and poured into a mixture of methanol and water. The crude polymer was filtered, washed with excess methanol, and dried. The crude polymer was further purified by redissolving the polymer into THF and reprecipitating in methanol several times to give PF-mCzP-mOXDP (431 mg, 38%) ¹H NMR (300 MHz, CDCl₃): δ 8.46 (s, 2H), 8.25-8.10 (m, 6H), 7.94-7.71 (m, 14H), 7.61-7.48 (m, 7H), 7.37 (t, 2H, J=6.6 Hz), 2.12 (bs, 8H), 1.11 (bs, 32H), 0.75 (m, 12H).

### Films of the Invention Comprising Host Compounds of the Invention and Phosphorescent Emitters

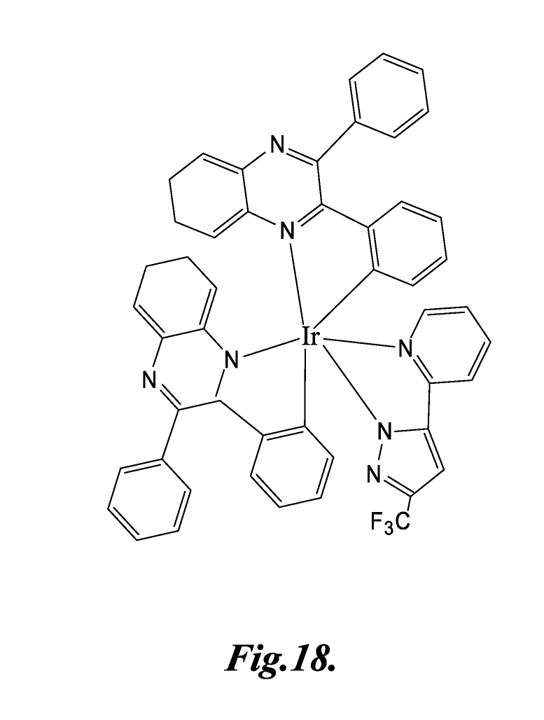

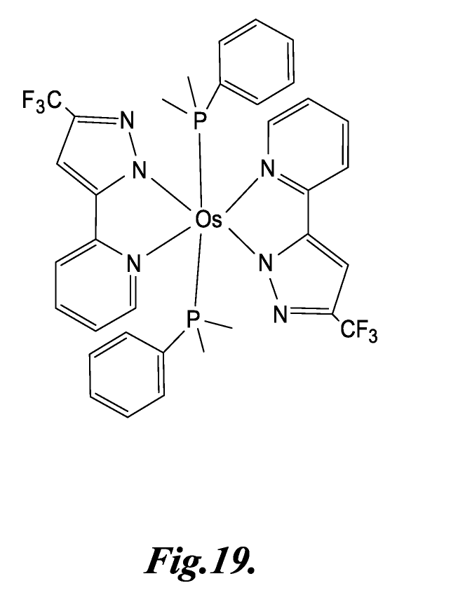

Films of the invention were made by dissolving host compounds of the invention and phosphorescent emitter guest compounds in a suitable solvent. PL measurements were made on a thin guest-host film made on a glass slide. An exemplary red-emitting phosphorescent guest material is CHY-2r-pz(CF₃) (“Ir-2R”), as illustrated in FIG. 18. The absorbance and PL spectrum of Ir-2R is graphically illustrated in FIG. 5. The absorption of Ir-2R is strong in the spectral region around 400 nm, the region where the emissive polyfluorenes/polyphenylene (PF/PP)-type host materials have the strongest PL emission. A second exemplary red-emitting phosphorescent guest material is Os-2, as illustrated in FIG. 19. An overlay of the PL emission of PF-mCzP-mOXDP with the absorption of Os-2 is illustrated in FIG. 4. The emission and absorption peaks show strong overlap, indicating favorable conditions for energy transfer between host and guest.

The transfer of energy between host and guest is illustrated in FIG. 6 with the representative host compound of the invention, PF-mCzP-mOXDP, and the red phosphorescent emitter Os-2. The PL emission spectra of the guest-host blends shows two emission bands: the host, with a maximum near 425 nm and the guest, with a maximum near 650 nm. Even at a the highest guest-doping-level (10 wt %), emission is still seen from the host; however the majority of the PL emission of the host is transferred to the guest at higher guest-doping-levels. The transfer of energy between guest and host during host PL is illustrative of the emission-absorption means of energy transfer from guest to host.

Charge-trapping is the second means for exciting the triplet-state of a guest phosphorescent molecule in a host. Because electrons and holes are needed in the charge-trapping mechanism, OLED devices are required to enable charge-trapping phosphorescence of a guest material. Charge-trapping can be detected (and distinguished from energy transfer) by analyzing the drive voltage dependence of an OLED on the concentration of the host phosphorescent emitter, as described in Holmes, et al., Applied Physics Letters 83:3818 (2003). Additionally, the lack of a host emission peak even at very low (<1%) guest doping levels is evidence of a charge-trapping mechanism. The spectra of an energy transfer-type OLED device, comprising an emissive film of Ir-2R as a guest in PF-mCzP, is illustrated in FIG. 8. Of particular importance is the low-guest-concentration peak near 425 nm as a result of EL of the host compound. Characterization data for the Ir-2R/PF-mCzP devices used to generate the data illustrated in FIG. 8 are summarized in Table 3.

TABLE 3
Device data for OLEDs incorporating representative films of the invention
comprising PF-mCzP and guest-emitter Ir-2R.

| Content of | | | | | | |
|---|---|---|---|---|---|---|
| Ir-2R in | | | LEₘₐₓ | Bₘₐₓ | V_{Bmax} | J_{Bmax} |
| PF-mCzP | Vₒₙ (V) | ηₘₐₓ (%) | (cd/A) | (cd/m²) | (V) | (mA/cm²) |
| 5% | 7.5 | 2.84 | 1.2 | 2350 | 19.5 | 202 |
| 2.5% | 7.5 | 2.55 | 1.05 | 1960 | 18.5 | 213 |
| 0.31% | 7 | 0.71 | 0.311 | 625 | 16 | 218 |

Vₒₙ: Turn-on voltage
ηₘₐₓ: Maximum external quantum efficiency
Bₘₐₓ: Maximum brightness
LE: Luminous efficiency
J_{Bmax}: Current density

Charge-trapping emissive layer OLED devices were made using Os-2 as a guest in PF-mCzP-mOXDP, with a device EL spectrum illustrated in FIG. 7. Characteristics of the devices used to generate the data illustrated in FIG. 7 are summarized in Table 4.

TABLE 4
Device data for OLEDs incorporating representative films of the invention
comprising PF-mCzP-mOXDP and guest-emitter Os-2.

| Content of | | | | | | | |
|---|---|---|---|---|---|---|---|
| Os-2 in | | | | | | | |
| PF-mCzP- | | ηₘₐₓ | Bₘₐₓ | PE | LE | CIE | |
| mOXDP | Vₒₙ (V) | (%) | (cd/m²) | (lm/W) | (cd/A) | x | y |
| 10% | 7 | 4.53 | 2350 | 0.91 | 2.87 | 0.67 | 0.33 |
| 5% | 8 | 4.28 | 6280 | 0.60 | 2.68 | 0.67 | 0.33 |
| 2.5% | 4.5 | 4.67 | 8230 | 1.19 | 3.06 | 0.67 | 0.33 |
| 1.25% | 5 | 3.57 | 8460 | 0.98 | 2.53 | 0.66 | 0.35 |
| 0.31% | 5.5 | 2.13 | 2230 | 0.74 | 1.59 | 0.53 | 0.35 |

Vₒₙ: Turn-on voltage
ηₘₐₓ: Maximum External quantum efficiency
Bₘₐₓ: Maximum brightness
LE: Luminous efficiency
PE: Power efficiency

Both the voltage dependence on concentration of guest emitter and the lack of a host EL peak show that a charge-trapping mechanism of operation is in effect for the Os-2 in PF-mCzP-mOXDP devices.

### Blue Phosphorescence from a Guest-Host Device of the Invention

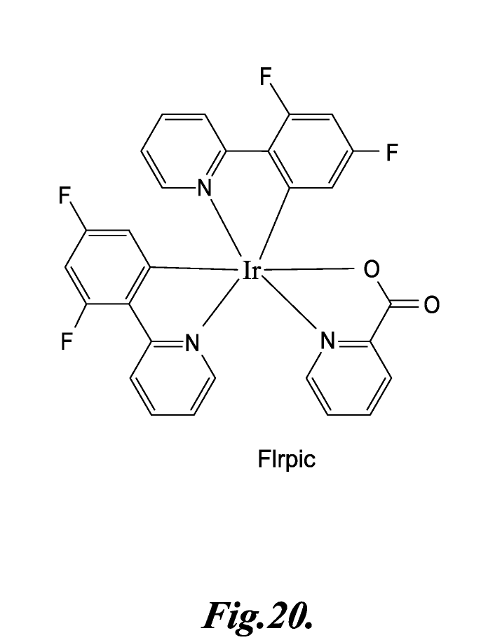

Compounds and films of the invention enable guest phosphorescence. The high triplet energy-levels of compounds of the invention help to facilitate energy transfer to high-energy blue phosphorescent guest compounds, as well block phosphorescent quenching. An exemplary blue phosphorescent OLED device uses a guest emitter, FIrpic (illustrated in FIG. 20), and a representative compound of the invention, PP-mCzP-mOXDP, as illustrated in the EL spectrum of FIG. 9. The high-energy emission of the phosphorescent guest indicates that the triplet energy-level of the host is sufficiently high to host the guest emitter without quenching the guest's phosphorescence.

### Branched Compounds of the Invention for Use as Host Materials

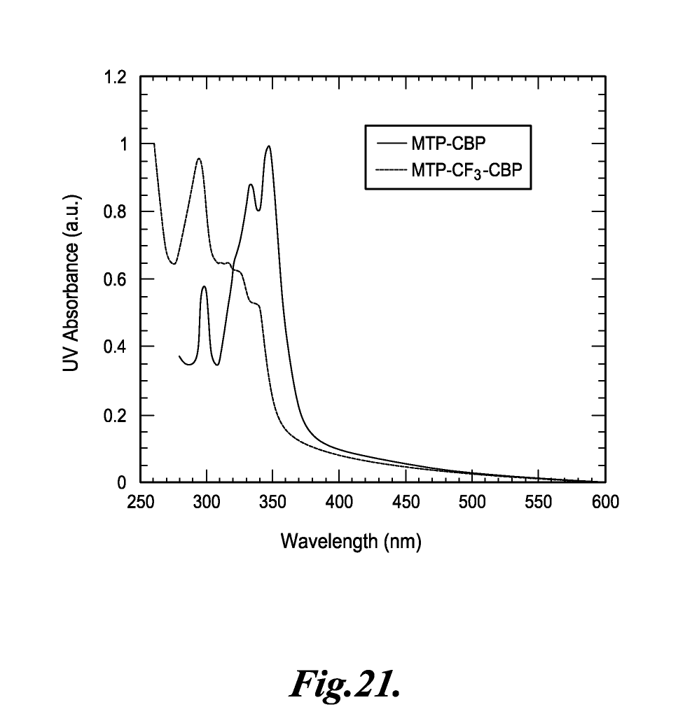

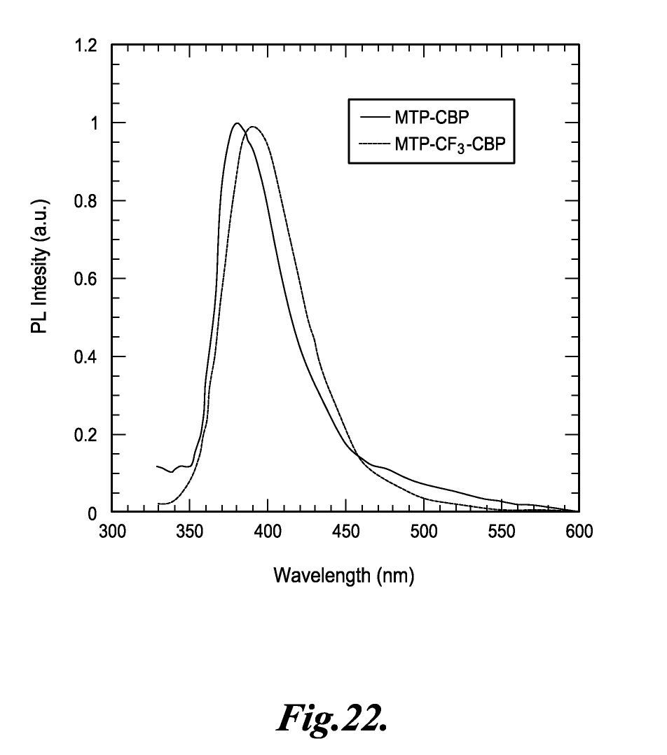

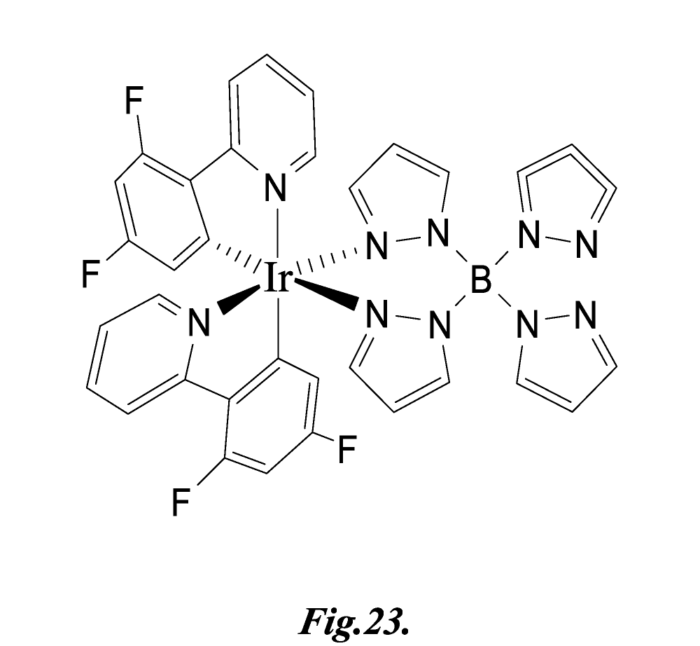

Branched compounds representative of the invention are illustrated in FIG. 3. The UV-Vis spectra of representative branched compounds are graphically illustrated in FIG. 21 and their PL spectra are graphically illustrated in FIG. 22. As shown in the UV-Vis spectra (FIG. 21), introducing two trifluoromethane onto the biphenyl of CBP results in the blue-shift of the UV absorption due to the twisting of the biphenyl. The bandgap of the branched compounds, calculated from the band edge of the absorption spectra, is 3.3 eV for MTP-CBP and 3.5 eV for MTP-CF3-CBP, respectively. The introduction of CF₃ groups onto the charge-transport moiety slightly red-shifts the PL compared to the non-CF₃ moiety. Branched compounds of the invention were used in OLED devices as hosts for the blue emitter FIr6, illustrated in FIG. 23. The exemplary OLEDs had the structure ITO/PEDOT:PSS/EL/TPBI/CsF/Al. TPBI (1,3,5-tris(N-phenylbenzimidazol-2-yl)benzene), was synthesized according to Applied Physics Letters, 74, 865 (1999). Device results are graphically illustrated in FIG. 10 and device characterization is shown in Table 5.

TABLE 5
OLED characterization for devices with an emissive layer comprising
branched compounds of the invention and the blue emitter FIr6.

| | | | Bₘₐₓ | | |
|---|---|---|---|---|---|
| EL layer | Vₒₙ (V) | ηₘₐₓ (%) | (cd/m²) | PE (lm/W) | LE (cd/A) |
| MTP- | 9.5 | 1.33 | 3040 | 0.87 | 2.97 |
| CBP:FIr6 = 90:10 | | | | | |
| MTP-CF₃- | 8 | 0.26 | 400 | 0.17 | 0.45 |
| CBP:FIr6 = 90:10 | | | | | |

Vₒₙ: Turn-on voltage
ηₘₐₓ: Maximum External quantum efficiency
Bₘₐₓ: Maximum brightness
PE: Power efficiency
LE: Luminous efficiency

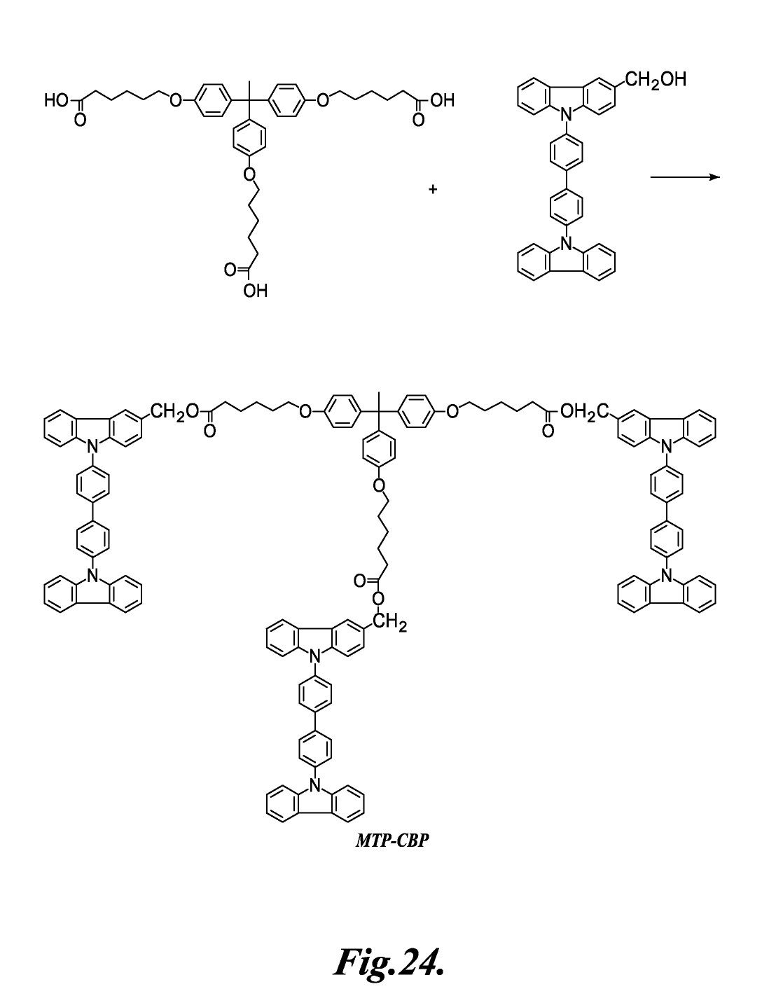

MTP-CBP. The synthesis of MTP-CBP is schematically illustrated in FIG. 24. To a solution of 1,1,1-tris(6-phenoxy-hexanoic acid methyl ester) ethane (115 mg, 0.17 mmol), CBP-CH₂OH (302 mg, 0.58 mmol), and 4-(dimethylamino)pyridine (DMAP, 21 mg, 0.17 mmol) in THF (20 mL) was added 1-[3-(dimethylamino)propyl]-3-ethyl carbodiimide hydrochloride (EDC, 121 mg, 0.63 mmol). After stirring at room temperature for 1 h, methylene chloride (10 mL) and a small amount of DMF (2 drops) were added into the suspension, and the reaction was heated to 50° C. overnight. The solvent was evaporated under reduced pressure. The resulting solid was re-dissolved in methylene chloride, washed with water, dried with Na₂SO₄, and then concentrated. The crude product was purified by column chromatography (2% ethyl acetate/methylene chloride) to afford MTP-CBP as a white solid (196 mg, 54%). ¹H NMR (300 MHz, CDCl₃): δ 8.15 (t, 12H, J=8.1 Hz), 7.89 (dd, 12H, J=8.5 Hz, J=2.0 Hz), 7.71-7.654 (m, 13 H), 7.51-7.43 (m, 24 H), 7.30 (t, 8H, J=6.5 Hz), 6.92 (dd, 6H, J=7.0 Hz, J=1.5 Hz), 6.71 (dd, 6H, J=9.0 Hz, J=2.0 Hz), 5.30 (s, 6H), 3.85 (t, 6H, J=6.5 Hz), 3.39 (t, 6H, J=7.0 Hz), 2.04 (s, 3H), 1.77-1.69 (m, 12H), 1.55-1.48 (m, 6H).

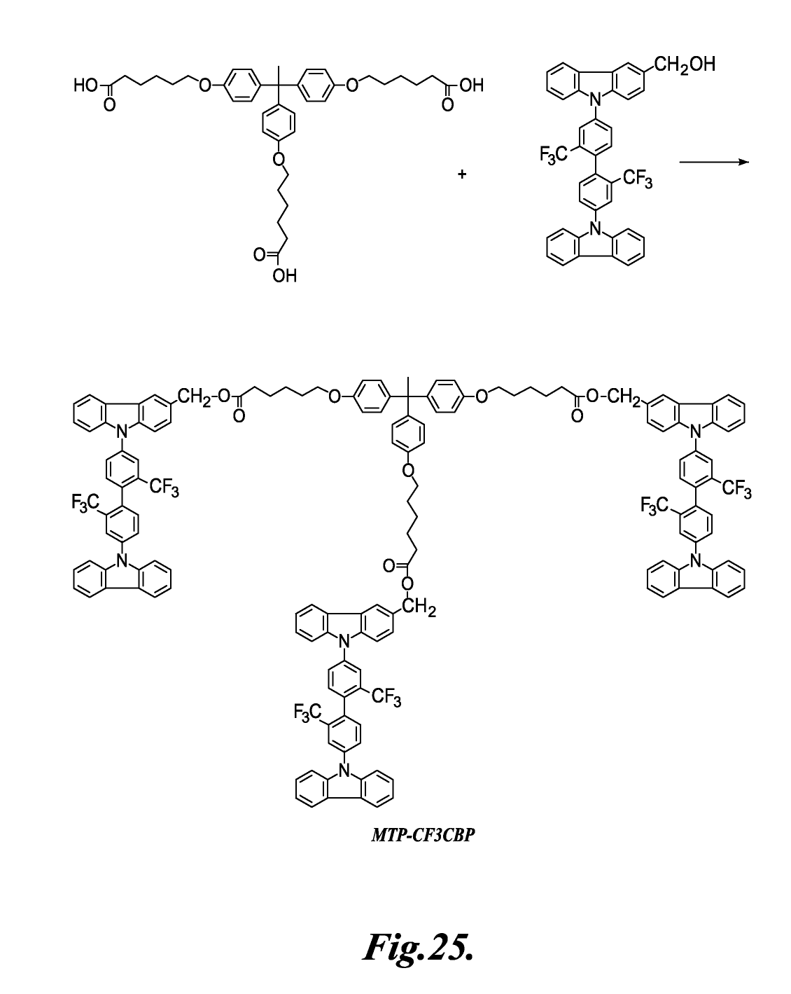

MTP-CF3CBP. The synthesis of MTP-CBP is schematically illustrated in FIG. 25. To a solution of 1,1,1-tris(6-phenoxy-hexanoic acid methyl ester) ethane (100 mg, 0.15 mmol), CBP-CH₂OH (335 mg, 0.51 mmol), and 4-(dimethylamino)pyridine (DMAP, 18 mg, 0.15 mmol) in THF (20 mL) was added 1-[3-(dimethylamino)propyl]-3-ethyl carbodiimide hydrochloride (EDC, 106 mg, 0.56 mmol). After stirring at room temperature for 1 h, methylene chloride (10 mL) and a small amount of DMF (2 drops) were added into the suspension, and the reaction was heated to 50° C. overnight. The solvent was evaporated under reduced pressure. The resulting solid was re-dissolved in methylene chloride, washed with water, dried with Na₂SO₄, and then concentrated. The crude product was purified by column chromatography (methylene chloride) to afford MTP-CBP as a white solid (284 mg, 74%). ¹H NMR (300 MHz, CDCl₃): δ 8.20 (t, 12H, J=7.2 Hz), 8.06 (d, 6H, J=8.1 Hz), 7.87 (t, 6H, J=9.6 Hz), 7.70 (d, 6H, J=8.4 Hz), 7.53-7.50 (m, 21H), 7.40-7,35 (m, 12H), 6.96 (d, 6H, J=9.9 Hz), 6.74 (d, 6H, J=9.0 Hz), 5.34 (s, 6H), 3.91 (t, 6H, J=6.6 Hz), 2.43 (t, 6H, J=7.5 Hz), 2.08 (s, 311), 1.86-1.70 (m, 12H), 1.54-1.50 (m, 6H).

### Example 2

### Light-Emitting Device Fabrication and Characterization

Light-emitting devices of the present invention are illustrated in FIGS. 11 and 12. FIG. 11 illustrates the most basic device structure of the invention. The device incorporates a film made of the compounds of the invention intermediate two electrodes. A more complex device structure can incorporate hole-transport layers, electron-transport layers, hole and electron-blocking layers, and charge-injection-enhancing layers adjacent to the electrodes. A typical complex device of the invention is illustrated in FIG. 12. Devices were fabricated on indium tin oxide (ITO)-coated glass substrates. The substrates were ultrasonicated sequentially in detergent, deionized water, 2-propanol, and acetone and were treated with O₂ plasma for 10 min before use. A layer of thermally-crosslinkable precursor, PS-TPD-TFV, in 1,2-dichloroethane with the concentration of 5 mg/mL was spin-coated onto the ITO and was thermally cross-linked at 235° C. for 40 min under argon to form a solvent-resistant layer. Optionally, a layer of commercial available polyethylene dioxythiophene polystyrene sulfonate (PEDOT:PSS, Bayer AG) film was spin-coated on the ITO or solvent-resistant layer, and cured at 125° C. for 10 min. A hole-transport layer (HTL) was formed by spin-coating a solution of PVK in 1,2-dichloroethane on top of the PEDOT:PSS layer. The electroluminescent (EL) layer was then spin-coated on top of the cross-linked PS-TPD-TFV layer, PEDOT:PSS layer, or PEDOT:PSS/PVK bilayer. In a vacuum below 1×10⁻⁶ torr, a layer of TPBI with thickness of 25 nm was sublimed. Cesium fluoride (CsF) with a thickness of 1 nm and 200 nm of Al were evaporated subsequently as a cathode.

Device testing was carried out in air at room temperature. EL spectra were recorded using an Oriel Instaspec IV spectrometer with a CCD detector. Current-voltage (I-V) characteristics were measured on a Hewlett-Packard 4155B semiconductor parameter analyzer. The power of EL emission was measured using a calibrated Si photodiode and a Newport 2835-C multifunctional optical meter. Photometric units (cd/m²) were calculated using the forward output power together with the EL spectra of the devices under assumption of the emission's Lambertian space distribution. The CIE coordinates were measured with the PR-650.

Example 3

### Synthesis of the Hole-Transporting Material PS-BTPD-PFCB

One-pot synthesis of crosslinkable hole-transporting side-chain polymer PS-BTPD-TFV.

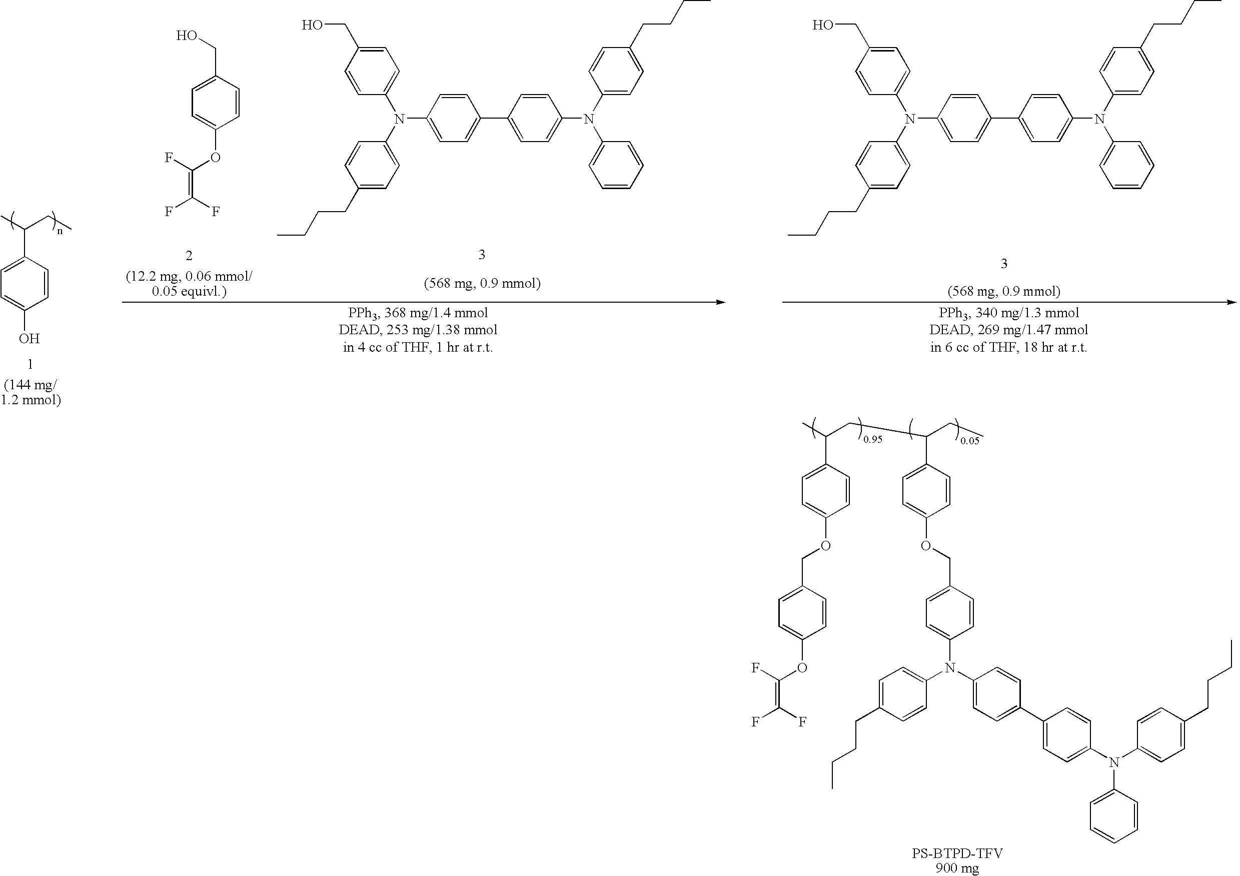

To 4.0 cc of freshly distilled THF was added poly(4-vinylphenol) (1, 144 mg, 1.2 mmol), triphenylphosphine (368 mg, 1.4 mmol), compound 2 (12.2 mg, 0.06 mmol/0.05 equivalent), and compound 3 (568 mg, 0.9 mmol). The resultant solution was stirred at room temperature under nitrogen atmosphere for several minutes, followed by the dropwise addition of the diethyl azodicarboxylate liquid (DEAD, 253 mg, 1.38 mmol). The reaction mixture was allowed to keep at room temperature for 1 hr. Then second batch of compound 3 (568 mg, 0.9 mmol) and triphenylphosphine (340 mg, 1.3 mmol) were added to the reaction mixture with 6.0 cc of dry THF and DEAD (269 mg, 1.47 mmol, dropwise) by the same sequence. The reaction mixture was allowed to keep at room temperature for extra 18 hr. The crude product of PS-BTPD-TFV was purified by three-time re-precipitation from its THF (and/or CH₂Cl₂) solution into stirring methanol to afford 900 mg of yellow solid.

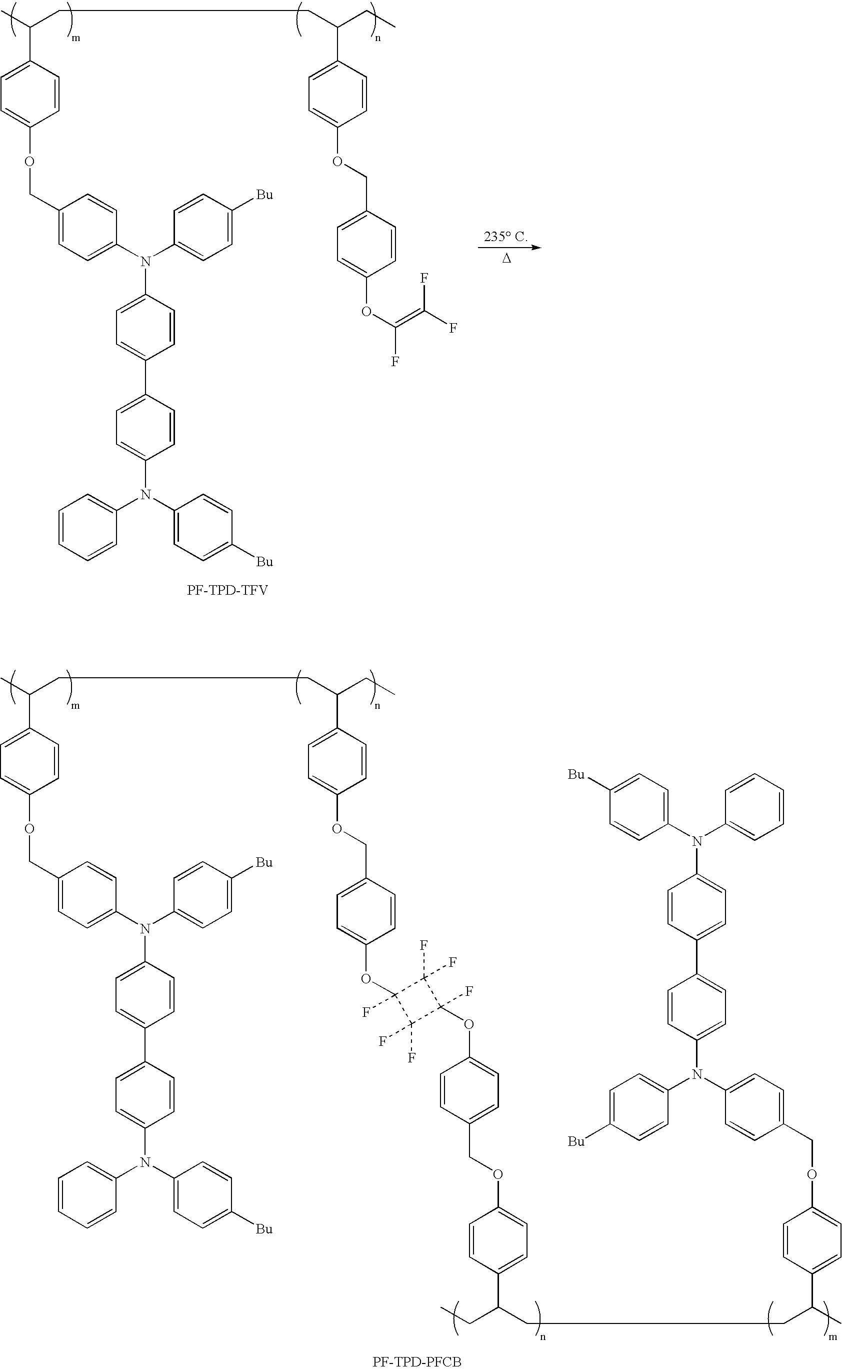

While illustrative embodiments have been illustrated and described, it will be appreciated that various changes can be made therein without departing from the spirit and scope of the invention.

The embodiments of the invention in which an exclusive property or privilege is claimed are defined as follows:

1. A polymer having a ground state to singlet excited state energy gap of from about 3.3 eV to about 3.5 eV, and a triplet energy greater than about 2.6 eV.

2. The polymer of claim 1 having an emission wavelength maximum of from about 360 nm to about 420 nm.

3. A polymer having the formula: 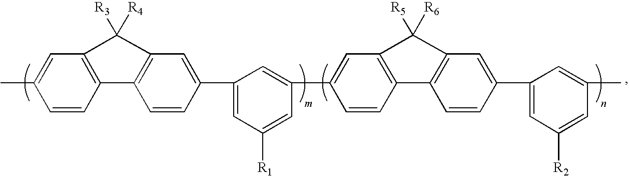
wherein R₁ and R₂ are independently selected from substituted and unsubstituted carbazole, substituted and unsubstituted thiophene, substituted and unsubstituted triphenyl amine, substituted and unsubstituted oxadiazole, substituted and unsubstituted triazine, substituted and unsubstituted benzothiadiazole, cyano, substituted and unsubstituted pyridine, substituted and unsubstituted quinoline, and substituted and unsubstituted quinoxaline;
wherein R₃, R₄, R₅, and R₆ are independently selected from branched and straight-chain alkyl groups having from one to twenty carbon atoms or branched and straight-chain alkoxy groups having from one to twenty carbon atoms; and
m is an integer from 0 to about 60;
n is an integer from 0 to about 60; and
m+n≧1.

4. The polymer of claim 3, wherein m is 0.

5. The polymer of claim 3, wherein n is 0.

6. The polymer of claim 3, wherein the ratio of m:n is about 1:1.

7. The polymer of claim 3, wherein R₁ is a carbazole.

8. The polymer of claim 3, wherein R₂ is an oxadiazole.

9. The polymer of claim 3, wherein R₃, R₄, R₅, and R₆ are nC₆H₁₃.

10. A polymer having the formula: 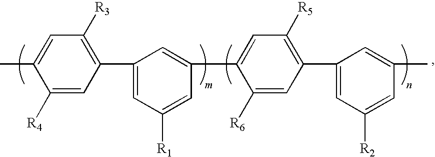
wherein R₁ and R₂ are independently selected from substituted and unsubstituted carbazole, substituted and unsubstituted thiophene, substituted and unsubstituted triphenyl amine, substituted and unsubstituted oxadiazole, substituted and unsubstituted triazine, substituted and unsubstituted benzothiadiazole, cyano, substituted and unsubstituted pyridine, substituted and unsubstituted quinoline, and substituted and unsubstituted quinoxaline;
wherein R₃, R₄, R₅, and R₆ are independently selected from branched and straight-chain alkyl groups having from one to twenty carbon atoms or branched and straight-chain alkoxy groups having from one to twenty carbon atoms; and
m is an integer from 0 to about 60;
n is an integer from 0 to about 60; and
m+n≧1.

11. The polymer of claim 10, wherein m is 0.

12. The polymer of claim 10, wherein n is 0.

13. The polymer of claim 10, wherein the ratio of m:n is about 1:1.

14. The polymer of claim 10, wherein R₁ is a carbazole.

15. The polymer of claim 10, wherein R₂ is an oxadiazole.

16. The polymer of claim 10, wherein R₃, R₄, R₅, and R₆ are nC₈H₁₇.

17. A compound having the formula
(E-L)ₙX
wherein n is 0, 1, 2, 3, or 4;
wherein X is an alkyl, heteroalkyl, or aryl core that is linked to charge-transporting moiety E by linker L; and
wherein E independently at any occurrence is the same or different from any other E in the compound.

18. A compound having the formula: 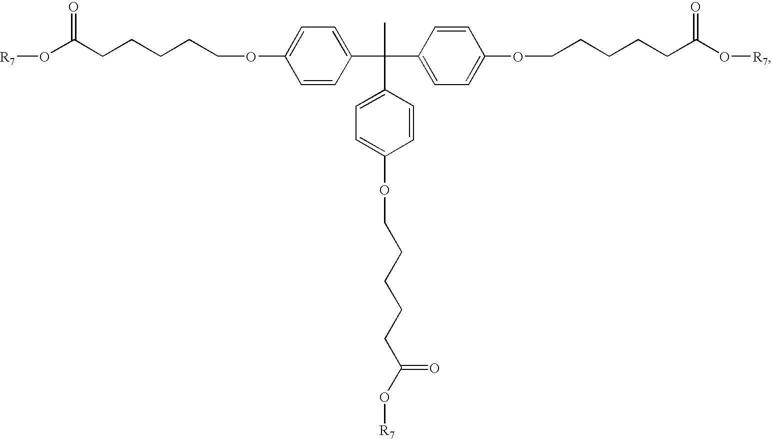
wherein R₇ is selected from the group consisting of

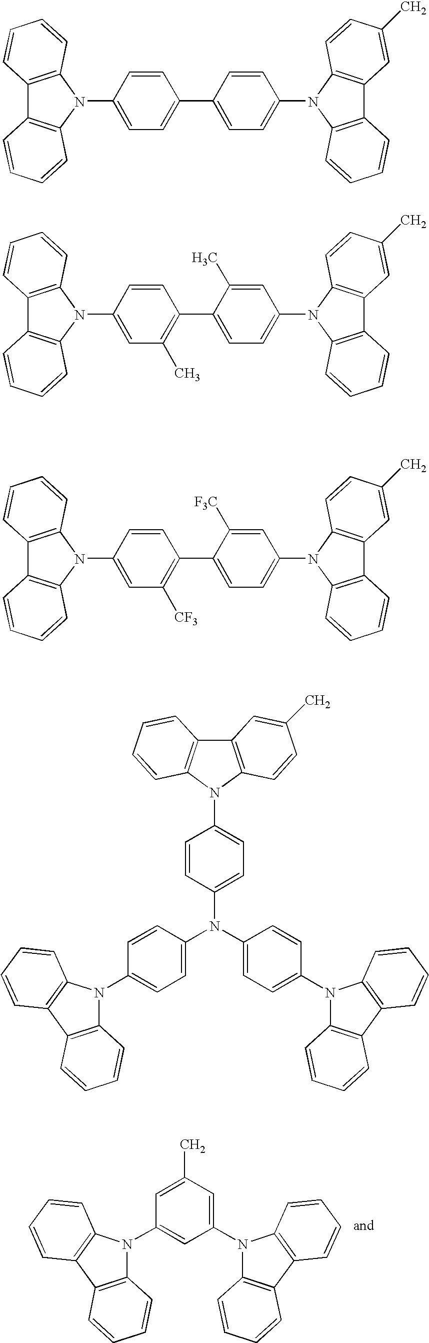

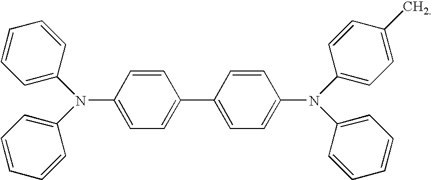

19. A film, comprising a compound of claim 1 and a first phosphorescent compound.

20. An electroluminescent device, comprising:
(a) a first electrode,
(b) a second electrode, and
(c) a film intermediate the first and second electrodes, wherein the film comprises a compound of claim 1 and a first phosphorescent compound.
